(19) United States
(12) Patent Application Publication
(10) Pub. No.: US 2016/0045168 A1
(43) Pub. Date: Feb. 18, 2016

(54) EARLY WARNING SCORE METHODOLOGIES

(71) Applicant: ALLSCRIPTS SOFTWARE, LLC, Chicago, IL (US)

(72) Inventors: Lee STORER, Middlewich (GB); Simon POTTER, Wigan (GB); Franck SCHMIDLIN, Chester (GB); Peter Emard FRIEBEL, Singapore (SG)

(21) Appl. No.: 14/588,151

(22) Filed: Dec. 31, 2014

Related U.S. Application Data
(60) Provisional application No. 62/036,182, filed on Aug. 12, 2014.
(60) Provisional application No. 62/036,299, filed on Aug. 12, 2014.

(51) Int. Cl. A61B 5/00; A61B 5/1455; G06F 19/00; A61B 5/021; A61B 5/024; A61B 5/08; A61B 5/01

(52) U.S. Cl. CPC A61B 5/7275; A61B 5/08; A61B 5/14552; A61B 5/01; A61B 5/021; A61B 5/024; G06F 19/322; G06F 19/3431

(57) ABSTRACT

A computer network includes computing devices and a server system having an electronic health records (EHR) application. The server system includes a logic module accessible by the EHR application and providing logic by which an early warning score for a patient is calculated. The score is calculated based on, at least in part, data from an electronic patient record that is accessed by the EHR application. The EHR application communicates the calculated early warning score to a networked device, which in turn displays the calculated early warning score on a screen for view by a user. The same networked device preferably is used to make one or more observations of the patient that are recorded in the electronic patient record, and based on which the score is calculated. Whether the score is increasing, decreasing, or remaining the same over observation intervals preferably is determined and displayed with the score.

### CROSS-REFERENCE TO RELATED APPLICATION

The present application is a U.S. nonprovisional patent application of, and claims priority under 35 U.S.C. §119(e) to, each of U.S. provisional patent application 62/036,182, filed Aug. 12, 2014, which provisional patent application is incorporated by reference herein; and U.S. provisional patent application 62/036,299, filed Aug. 12, 2014, which provisional patent application is incorporated by reference herein. The present application hereby further incorporates herein by reference the entire disclosure of the Appendix attached hereto, which represents the disclosure of these two provisional applications.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

The present invention generally relates to the use of an early warning score in a medical context.

In healthcare, early warning score (EWS) methodologies are frequently used by hospital nursing and medical staff and emergency medical services as a simple guide to quickly determine the degree of illness or injury of a patient. It is often based on data derived from physiological readings (such as blood oxygen saturation, systolic blood pressure, heart rate, respiratory rate, body temperature, etc.) and other observations (such as level of consciousness and pain level). Typically, resulting observations are compared to a normal range to generate a single composite score.

EWS methodologies can be used to inform best medical practices, for example, an early warning score can prompt a nurse to call an attending physician or increase the frequency of vital sign observations.

Existing EWS calculations are either done manually, through a paper based score card, or electronically, using a hardcoded algorithm that requires the attending nurse to input the vital signs and other observations.

Both paper based score cards and hard coded algorithms make the EWS calculation a blunt instrument that will raise false alarms. For example, a patient on specific heart medicine might experience an elevated heart rate that would score them unnecessarily high.

Although there are advantages in adopting standardized EWS calculations, these do not apply effectively across all patient groups (e.g., pediatrics, oncology, etc.), again resulting in lower accuracy and increased false alarms.

EWS false alarms can cause alert fatigue, and cause an attending practitioner to start second guessing the scoring and not reacting appropriately, which can potentially cause patient safety issues.

Available literature on EWS adoption identifies alarm fatigue as one of the factors impeding the wider adoption of EWS methodologies as an effective means to detect deteriorating patients.

One or more needs exist for improvement in early warning score methodologies, including improving the accuracy in such methodologies so that less false alarms are experienced and less alert fatigue results. These, and other needs, are addressed by one or more aspects of the present invention.

### SUMMARY OF THE INVENTION

The present invention includes many aspects and features. Moreover, while many aspects and features relate to, and are described in, the context of providing healthcare services at a hospital, the present invention is not limited to use only in this context, as will become apparent from the following summaries and detailed descriptions of aspects, features, and one or more embodiments of the present invention.

Accordingly, one aspect of the present invention relates to a configurable logic module for an electronic health records (EHR) application that is utilized to deliver early warning score (EWS) calculations.

In one or more preferred implementations, a logic module takes into account a patient's medications and treatments in EWS calculations.

In one or more preferred implementations, a logic module takes into account a patient's medical history and specific patient exceptions, such as, for example, a known history of high blood pressure, in EWS calculations.

In one or more preferred implementations, a logic module delivers EWS calculations that meet the specific requirement of a facility or a unit.

In one or more preferred implementations, a logic module provides EWS calculations tailored to one or more specific patient groups.

In one or more preferred implementations, a logic module provides EWS calculations tailored to one or more EWS standards.

In one or more preferred implementations, a logic module provides EWS calculations tailored to one or more specific patient groups and to one or more EWS standards.

In another aspect of the invention, a computer network provides early warning scores for a plurality of patients, which scores are used by medical personnel to identify degrees of illness or injury of patients. In this regard, the computer network comprises: one or more networked devices desktop computers and one or more mobile computing devices; a server system comprising one or more computers and arranged in electronic communication with the one or more networked desktop computers and one or more mobile computing devices. The server system includes a non-transient computer readable medium in which an electronic health records (EHR) application is stored in the form of computer-executable code, by which EHR application each of the computers and mobile computing devices can access, over the computer network, patient data in electronic patient records maintained for patients. The server system includes a non-transient computer readable medium in which one or more logic modules are stored, each logic module being accessible by the EHR application and each logic module providing logic by which a task is performed by or for the EHR application, the logic of each logic module being alterable without alteration to the computer-executable code of the EHR application. In accordance with this aspect, a logic module provides logic by which an early warning score is calculated, the logic including calculating an early warning score based on, at least in part, data from an electronic patient record that is accessed by the EHR application for calculating such early warning score. The EHR application is adapted to communicate a calculated early warning score to at least one of the one or more networked devices, which in turn is adapted to display the calculated early warning on a screen for view by a user.

In a feature of this aspect, the data from the electronic patient record on which data the early warning score is calculated includes a patient respiration rate.

In another feature, the data from the electronic patient record on which data the early warning score is calculated includes an oxygen saturation.

In another feature, the data from the electronic patient record on which data the early warning score is calculated includes an oxygen device flow rate.

In another feature, the data from the electronic patient record on which data the early warning score is calculated includes a temperature of the patient.

In another feature, the data from the electronic patient record on which data the early warning score is calculated includes a blood pressure of the patient.

In another feature, the data from the electronic patient record on which data the early warning score is calculated includes a heart rate of the patient.

In another feature, the data from the electronic patient record on which data the early warning score is calculated includes a level of consciousness of the patient.

In another feature, the data from the electronic patient record on which data the early warning score is calculated includes a level of pain experienced by the patient.

In another feature, the logic module is configured to calculate the early warning scores.

In another feature, the EHR application is configured to calculate the early warning scores using the logic provided by the logic module.

In another feature, the computer network is implemented by a plurality of healthcare facilities, and a single logic module provides different logic for calculating early warning scores for patients of at least two of the healthcare facilities based on identification of the healthcare facility of a patient.

In another feature, the computer network is implemented by a plurality of medical services departments of a healthcare facility, and a single logic module provides different logic for calculating early warning scores for patients of at least two of the medical services departments based on identification of the medical services department of a patient.

In another feature, a single logic module provides different logic for calculating early warning scores for patients based on an identification of a patient group of a patient from a plurality of different patient groups.

In another feature, a single logic module provides different logic for calculating early warning scores for patients based on an identification of an EWS standard from a plurality of different EWS standards.

In another feature, the computer network is implemented by a plurality of healthcare facilities, and the computer network further comprises a plurality of logic modules each corresponding to a respective one of the plurality of healthcare facilities, at least two of the logic modules providing different logic for calculating early warning scores for patients based on the healthcare facility of the patient.

In another feature, the computer network is implemented by a plurality of medical service departments of a healthcare facility, and the computer network further comprises a plurality of logic modules each corresponding to a respective one of the medical services departments of the healthcare facility, at least two of the logic modules providing different logic for calculating early warning scores for patients based on the medical service department of the patient.

In another feature, the computer network further comprises a plurality of logic modules each corresponding to a respective patient group, at least two of the logic modules providing different logic for calculating early warning scores for patients.

In another feature, the computer network further comprises a plurality of logic modules each corresponding to a respective EWS standard, at least two of the logic modules providing different logic for calculating early warning scores for patients.

In another feature, the logic provided by the logic module, when calculating an early warning score of a patient, takes into account a medical history of the patient.

In another feature, the logic provided by the logic module, when calculating an early warning score of a patient, takes into account one or more exception orders for the patient.

In another feature, the logic provided by the logic module, when calculating an early warning score of a patient, takes into account one or more current medications of the patient.

In another feature, the logic provided by the logic module, when calculating an early warning score of a patient, takes into account one or more current treatments of the patient.

In another feature, the server system includes non-transient computer readable medium in which a database of the electronic patient records is stored, which database is accessible by the EHR application for reading and writing patient data to the electronic patient records.

In another feature, the logic module further provides logic by which a determination is made whether the early warning score of the patient is trending higher, trending lower, or remaining the same based on a comparison between the early warning score calculated and one or more early warning scores previously recorded in the electronic patient record of the patient, and the EHR application is adapted to communicate an indication of whether the early warning score of the patient is trending higher, trending lower, or remaining the same to at least one of the one or more networked devices, which in turn is adapted to display the indication on a screen for view by a user.

In another feature, a logic module comprises a script for performance of a specific task for or on behalf of the EHR Application.

In another feature, a logic module comprises an computer-executable program for performing a specific task for or on behalf of the EHR Application.

In another feature, the logic module is attached to computerized flowsheets of the EHR application.

In another aspect of the invention, a method of calculating in a computer network early warning scores for a plurality of patients, which scores are used by medical personnel to identify degrees of illness or injury of patients, comprises: receiving from networked devices for a plurality of patients, by an electronic health records (EHR) application, observation data for one or more observations made regarding the patients, and for each of the patients, recording in an electronic patient record of such patient the received observation data for such patient; accessing from a logic module, by the EHR application, logic for calculating an early warning score and, in accordance with such logic, accessing recorded observation data from an electronic patient record for calculating the early warning score based on the accessed observation data recorded in the electronic patient record; and communicating, by the EHR application, to one of the networked devices, for display to medical personnel, an early warning score calculated based on the accessed logic and accessed observation data recorded in the electronic patient record.

In a feature, the accessing step of is performed for a particular patient upon receiving and recording observation data for the particular patient from one of the networked devices, and the communicating step comprises communicating a calculated early warning score for such patient to the networked device from which the observation data is received.

In a feature of the invention, the method further comprises altering the logic of the logic module without altering code of the EHR application.

In a feature of the invention, the method further comprises calculating the early warning score by the EHR application based on the accessed observation data recorded in the electronic patient record.

In a feature of the invention, the method further comprises calculating the early warning score by the logic module based on the accessed observation data recorded in the electronic patient record.

In a feature of the invention, the method further comprises displaying the calculated early warning score on a display screen of the networked device to which the calculated early warning score is communicated.

In a feature of the invention, the logic module that is accessed by the EHR application provides the logic for calculating an early warning score for each of the plurality of patients.

In a feature of the invention, the method further comprises, in accordance with the logic for calculating the early warning score, accessing recorded data from the electronic patient record of the patient in addition to the recorded observation data that is accessed from the electronic patient record, and calculating the early warning score based on the observation data and the additional data that is accessed from the electronic patient record of the patient. The additional data that is accessed from the electronic patient record may comprise a medical history of the patient; may comprise a medical history of high blood pressure of the patient; may comprise one or more exception orders for the patient; may comprise one or more oxygen saturation exception orders for the patient; may comprise one or more heart rate exception orders for the patient; may comprise one or more respiration rate exception orders for the patient; may comprise one or more blood pressure exception orders for the patient; may comprise one or more current medications of the patient; may comprise one or more current treatments of the patient; and combinations thereof. The additional data also may indicate oxygen therapy of the patient, and calculating the early warning score then may include adjusting a blood oxygen saturation range for normalization of an observed blood oxygen saturation of the patient.

In a feature of the invention, the method further comprises in accordance with the logic for calculating the early warning score, accessing recorded data from the electronic patient record of the patient in addition to the recorded observation data that is accessed from the electronic patient record, wherein the additional data comprises one or more previous early warning scores; determining whether the current early warning score is trending higher, trending lower, or remaining the same over an observation interval; and communicating, by the EHR application, to one of the networked devices, for display to medical personnel, an indication of whether the current early warning score is trending higher, trending lower, or remaining the same.

In another aspect of the invention, a method for quickly and accurately identifying a degree of illness or injury of a patient comprises: (a) receiving, by a networked device, user input regarding one or more observations made regarding the patient; (b) communicating, by the networked device, the received user input to an electronic health records (EHR) application for recording thereof in an electronic patient record of the patient; (c) in response to performance of said step (b) by the networked device, (i) receiving, by the networked device, from the EHR application, a current early warning score for the patient based at least in part on the communicated user input when sufficient user input regarding one or more observations has been communicated for calculating a current early warning score for the patient, and (i) receiving, by the networked device, from the EHR application, a message that the user input communicated was insufficient for calculating a current early warning score for the patient when insufficient user input regarding one or more observations has been communicated for calculating a current early warning score for the patient; and (d) displaying, by the networked device for view by a user, (i) the current early warning score when received from the EHR application, and (ii) the message that the user input communicated was insufficient for calculating a current early warning score for the patient when such message is received from the EHR application.

In a feature, the user input regards respiration rate of the patient.

In a feature, the user input regards oxygen saturation of the patient.

In a feature, the user input regards oxygen device flow rate of the patient.

In a feature, the user input regards temperature of the patient.

In a feature, the user input regards systolic blood pressure of the patient.

In a feature, the user input regards heart rate of the patient.

In a feature, the user input regards a level of pain being experienced of the patient.

In a feature, the user input regards a level of consciousness exhibited by the patient.

Another aspect of the invention comprises software in the form of computer-executable instructions contained in a computer-readable medium which, when executed by a processor, performs a method in accordance with one or more aspects or features of the invention.

In another aspect, a configurable logic module for an electronic health records application—such as “Sunrise Clinical Manager” available from Allscripts Healthcare Solutions, Inc. and its subsidiaries or similar commercial offering—is utilized to deliver early warning score (EWS) calculations. The logic module preferably takes into account: a patient's medications and treatments in EWS calculations; a patient's medical history and specific patient exceptions, such as, for example, a known history of high blood pressure; the specific requirement of a facility or a unit within that facility; one or more specific patient groups; one or more EWS standards; and combinations of the foregoing.

Another aspect relates to a method for submitting multiple patient observations using asynchronous web service calls by utilizing a publish subscribe pattern for communications between a user interface, a submission engine, and web services. The method includes displaying, to a user via an electronic display associated with an electronic device, an interface of a healthcare software application configured to allow a user to trigger submission of patient observations; receiving, at the electronic device from the user via one or more input devices associated with the electronic device, input corresponding to an indication to effect submission of a plurality of patient observations; subscribing, by a user interface module of the healthcare software application, to published messages from a submission engine concerning the triggered submission, and communicating an indication of the triggering of the submission to the submission engine; subscribing, by the submission engine, to published messages from a submission handler concerning a first submission message, and communicating the first submission message including first one or more observations of the plurality of patient observations to the submission handler; generating, at the submission handler in response to the communicated first submission message, a first call to one or more web services; handling, at each of the one or more web services, the first call, and communicating, to the submission handler, a first indication of success; publishing, by the submission handler in response to the received first indication of success, a first success message; handling, at the submission engine, the published first success message, and determining that there is at least one additional observation to be submitted; determining, at the submission engine, whether a test pause setting is active; subscribing, by the submission engine, to published messages from a submission handler concerning a second submission message, and communicating the second submission message including one or more of the at least one additional observations to be submitted to the submission handler; generating, at the submission handler in response to the communicated second submission message, a second call to one or more web services; handling, at each of the one or more web services, the second call, and communicating, to the submission handler, a second indication of success; publishing, by the submission handler in response to the received second indication of success, a second success message; handling, at the submission engine, the published second success message, and determining whether there is another additional observation to be submitted; after determining that there are not any more additional observations to be submitted, determining, at the submission engine that all of the submissions were successful, and publishing, by the submission engine, a full success message; and handling, at the user interface module, the full success message, and, based thereon, displaying, to the user via the electronic display associated with the electronic device, an indication that patient observations were successfully submitted.

In a feature of this aspect, the electronic device comprises a phone.

In a feature of this aspect, the electronic device comprises a tablet.

In a feature of this aspect, the electronic device comprises an iOS device.

In a feature of this aspect, the electronic device comprises an iPhone.

In a feature of this aspect, the electronic device comprises an iPad.

In a feature of this aspect, the electronic device comprises an Android device.

In a feature of this aspect, the electronic device comprises a Windows mobile device.

In a feature of this aspect, the electronic device comprises a Windows 8 device.

In a feature of this aspect, the electronic device comprises a Microsoft Surface.

Another aspect relates to a method for submitting multiple patient observations using asynchronous web service calls by utilizing a publish subscribe pattern for communications between a user interface, a submission engine, and web services. The method includes displaying, to a user via an electronic display associated with an electronic device, an interface of a healthcare software application configured to allow a user to trigger submission of patient observations; receiving, at the electronic device from the user via one or more input devices associated with the electronic device, input corresponding to an indication to effect submission of a plurality of patient observations; subscribing, by a user interface module of the healthcare software application, to published messages from a submission engine concerning the triggered submission, and communicating an indication of the triggering of the submission to the submission engine; subscribing, by the submission engine, to published messages from a submission handler concerning a first submission message, and communicating the first submission message including first one or more observations of the plurality of patient observations to the submission handler; generating, at the submission handler in response to the communicated first submission message, a first call to one or more web services; handling, at each of the one or more web services, the first call, and communicating, to the submission handler, a first indication of success; publishing, by the submission handler in response to the received first indication of success, a first success message; handling, at the submission engine, the published first success message, and determining that there is at least one additional observation to be submitted; determining, at the submission engine, that a test pause setting is active, and, based thereon, subscribing to pause continue messages from the user interface module, and publishing a pause message; handling, at the user interface module, the pause message, and, based thereon, displaying, to the user via the electronic display associated with the electronic device, a pause modal; receiving, at the electronic device from the user via one or more input devices associated with the electronic device, input corresponding to interaction with the pause modal; publishing, based on the input corresponding to interaction with the pause modal, a pause continue message; handling, at the submission engine, the pause continue message and, in response thereto, subscribing, by the submission engine, to published messages from a submission handler concerning a second submission message, and communicating the second submission message including one or more of the at least one additional observations to be submitted to the submission handler; generating, at the submission handler in response to the communicated second submission message, a second call to one or more web services; handling, at each of the one or more web services, the second call, and communicating, to the submission handler, a second indication of success; publishing, by the submission handler in response to the received second indication of success, a second success message; handling, at the submission engine, the published second success message, and determining whether there is another additional observation to be submitted; after determining that there are not any more additional observations to be submitted, determining, at the submission engine that all of the submissions were successful, and publishing, by the submission engine, a full success message; handling, at the user interface module, the full success message, and, based thereon, displaying, to the user via the electronic display associated with the electronic device, an indication that patient observations were successfully submitted.

Another aspect relates to a method for submitting multiple patient observations using asynchronous web service calls by utilizing a publish subscribe pattern for communications between a user interface, a submission engine, and web services. The method includes displaying, to a user via an electronic display associated with an electronic device, an interface of a healthcare software application configured to allow a user to trigger submission of patient observations; receiving, at the electronic device from the user via one or more input devices associated with the electronic device, input corresponding to an indication to effect submission of a plurality of patient observations; subscribing, by a user interface module of the healthcare software application, to published messages from a submission engine concerning the triggered submission, and communicating an indication of the triggering of the submission to the submission engine; subscribing, by the submission engine, to published messages from a submission handler concerning a first submission message, and communicating the first submission message including first one or more observations of the plurality of patient observations to the submission handler; generating, at the submission handler in response to the communicated first submission message, a first call to one or more web services; handling, at each of the one or more web services, the first call, and communicating, to the submission handler, a first indication of success; publishing, by the submission handler in response to the received first indication of success, a first success message; handling, at the submission engine, the published first success message, and determining that there is at least one additional observation to be submitted; subscribing, by the submission engine, to published messages from a submission handler concerning a second submission message, and communicating the second submission message including one or more of the at least one additional observations to be submitted to the submission handler; generating, at the submission handler in response to the communicated second submission message, a second call to one or more web services; handling, at each of the one or more web services, the second call, and communicating, to the submission handler, a second indication of success; publishing, by the submission handler in response to the received second indication of success, a second success message; handling, at the submission engine, the published second success message, and determining whether there is another additional observation to be submitted; after determining that there are not any more additional observations to be submitted, determining, at the submission engine that all of the submissions were successful, and publishing, by the submission engine, a full success message; and handling, at the user interface module, the full success message, and, based thereon, displaying, to the user via the electronic display associated with the electronic device, an indication that patient observations were successfully submitted.

Another aspect relates to a computer network which includes computing devices and a server system having an electronic health records (EHR) application. The server system includes a logic module accessible by the EHR application and providing logic by which an early warning score for a patient is calculated. The score is calculated based on, at least in part, data from an electronic patient record that is accessed by the EHR application. The EHR application communicates the calculated early warning score to a networked device, which in turn displays the calculated early warning score on a screen for view by a user. The same networked device preferably is used to make one or more observations of the patient that are recorded in the electronic patient record, and based on which the score is calculated. Whether the score is increasing, decreasing, or remaining the same over observation intervals preferably is determined and displayed with the score.

Another aspect relates to a patient observations submission state engine that uses native asynchronous web service handling (e.g. that in iOS) and manages multiple submission transaction requests for a single patient observation session. Preferably, the transactions and their results are managed by the engine, and appropriate result events (success, partial failure and full failure) are pushed back to a user interface.

In addition to the aforementioned aspects and features of the present invention, it should be noted that the present invention further encompasses the various possible combinations and subcombinations of such aspects and features. Thus, for example, any aspect may be combined with an aforementioned feature in accordance with the present invention without requiring any other aspect or feature.

### BRIEF DESCRIPTION OF THE FIGURES

FIG. 1 schematically illustrates a computer network in which one or more preferred embodiments of the present invention are incorporated.

FIG. 2 illustrates components of the server system of the computer network of FIG. 1.

FIG. 3 illustrates steps of a method of one or more preferred embodiments of the invention.

FIG. 4 illustrates a sequence of communications in one or more preferred embodiments of the invention.

FIG. 5 illustrates steps of a method that, in one or more preferred embodiments, is performed through the sequence of communications illustrated in FIG. 4.

FIG. 6 illustrates steps of another method that, in one or more preferred embodiments, is performed through an alternative sequence of communications.

FIG. 7 illustrates steps of another method that is performed in one or more preferred embodiments through the sequence of communications illustrated in FIG. 4.

FIG. 8 illustrates steps of another method that is performed in one or more preferred embodiments through the sequence of communications illustrated in FIG. 4.

FIG. 9 illustrates steps of another method that is performed in one or more preferred embodiments through the sequence of communications illustrated in FIG. 4.

FIG. 10 illustrates steps of another method that is performed in one or more preferred embodiments through the sequence of communications illustrated in FIG. 4.

FIG. 11 illustrates a home screen of an iOS mobile device is shown with icons representing apps arranged thereon.

FIG. 12 illustrates a login box for authentication of a user on the iOS mobile device.

FIG. 13 illustrates a listing of patients on the iOS mobile device, for whom observations can be made by a user with the iOS mobile device.

FIG. 14 illustrates a user interface for receiving user input regarding observations made by a user for a specific patient.

FIG. 15 illustrates user input being received in the user interface of FIG. 14 regarding a respiration rate of a specific patient.

FIG. 16 illustrates user input being received in the user interface of FIG. 14 regarding a respiration rate of a specific patient, wherein the value for the respiration rate is outside of a normal range

FIG. 17 illustrates a secondary user interface that appears when the “Click to select” element of the user interface of FIG. 17 is selected by the user.

FIG. 18 illustrates the results of selecting “Nasal Cannulae” from the secondary user interface of FIG. 17.

FIG. 19 illustrates what occurs when the submit button is selected and there is an insufficient number of observations being submitted so as to calculate an early warning score based thereon.

FIG. 20 illustrates what occurs when the submit button is selected and the connection to the computer network has been lost.

FIG. 21 illustrates the transfer of submitted observations from the iOS mobile device to a “flowsheets” tabbed page within an EHR Application, which page is in the context of the identified patient.

FIG. 22 better illustrates the user interface of FIG. 21

FIG. 23 better illustrates the tabbed page of FIG. 21.

FIG. 24 illustrates the display to a user on iOS mobile device of a user interface for an early warning score calculated following submission, wherein the score is calculated to be a “0”.

FIG. 25 illustrates the display to a user on iOS mobile device of a user interface for an early warning score calculated following submission, wherein the score is calculated to be a “3”.

FIG. 26 illustrates the display to a user on iOS mobile device of a user interface for an early warning score calculated following submission, wherein the score is calculated to be a “17”.

FIG. 27 illustrates the display to a user on iOS mobile device of a user interface for an early warning score calculated following submission, wherein the score is calculated to be a “4”.

FIG. 28 illustrates the display to a user on iOS mobile device of a user interface wherein not enough observations have been made contemporaneously with the submission so as to be able to calculate a current early warning score.

FIG. 29 illustrates a portion of a screen shot of a user interface of an EHR Application, wherein an “Orders” tabbed page is displayed to a user that includes an “Order Summary” for a particular patient and, as shown, an exception order is revealed for Nursing that relates to the calculation of an early warning score for the particular patient.

FIG. 30 shows a flowchart illustrating a sequence of steps performed in accordance with a preferred embodiment of the invention.

### DETAILED DESCRIPTION

As a preliminary matter, it will readily be understood by one having ordinary skill in the relevant art (“Ordinary Artisan”) that the present invention has broad utility and application. As should be understood, any embodiment may incorporate only one or a plurality of the above-disclosed aspects of the invention and may further incorporate only one or a plurality of the above-disclosed features. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention in any claim of a patent issuing here from, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. §112, paragraph (f), no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

One or more preferred embodiments of the present invention are next described. The following description of one or more preferred embodiments is merely exemplary in nature and is in no way intended to limit the invention, its implementations, or uses.

In accordance with one or more preferred implementations, a configurable logic module for an electronic health records (EHR) application is utilized to deliver early warning score (EWS) calculations. In one or more preferred implementations, a logic module is a logic module for a clinical management application such as “Sunrise Clinical Manager” available from Allscripts Healthcare Solutions, Inc. and its subsidiaries. As will be appreciated, Allscripts' Sunrise Clinical Manager—or SCM—is built on top of a fully comprehensive electronic patient record (EPR), and SCM enables complex clinical workflows to be easily tailored through configurable modules, known as “medical logic modules” or MLMs—that have full access to the EPRs.

In one or more preferred implementations, a logic module—such as an MLM in implementations using SCM—takes into account a patient's medications and treatments in EWS calculations. For example, in one or more preferred implementations, a system may lower what is considered to be a normal blood oxygen saturation range for a patient receiving oxygen therapy.

In one or more preferred implementations, a logic module takes into account a patient's medical history and specific patient exceptions, such as, for example, a known history of high blood pressure, in EWS calculations.

In one or more preferred implementations, EWS calculation uses the following observations: respiration rate, oxygen saturation, oxygen device flow rate, temperature, systolic blood pressure, heart rate, and level of consciousness preferably using the AVPU scale—an acronym from “alert, voice, pain, unresponsive”—by which a first aider, ambulance crew or health care professional measures and records a patient's responsiveness.

In one or more preferred implementations, the EWS calculation is implemented by a single logic module that takes into account the following orders to offset the scoring of individual observations: oxygen therapy order minimum and maximum oxygen saturation; heart rate exception orders; respiration rate exception orders; and blood pressure orders.

In one or more preferred implementations, a logic module charts or tracks early warning score over time, allowing medical staff to view not just a patient's current score, but also whether the patient's score is increasing (e.g. patient is deteriorating) or decreasing (e.g. patient is improving) over a period of time.

One or more preferred implementations relate to a logic module which delivers EWS calculations that meet the specific requirement of a facility, or even a unit within that facility (oncology, emergency room, pediatrics, etc.).

In one or more preferred implementations, a logic module includes EWS calculations tailored to one or more specific patient groups and/or one or more EWS standards.

Preferably, EWS calculation functionality is provided in a logic module which can be easily maintained and modified without modifying the underlying clinical management application, thereby allowing healthcare providers to tailor their EWS calculations through try-inspect-adapt cycles or consensus methods much more easily than with a paper-based score card or a hard-coded electronic algorithm.

Preferably, EWS calculation functionality is provided in a logic module which can be attached to flowsheets or called by dedicated software components, allowing the calculation to be shared across components and thus providing consistency of result across a range of applications.

In one or more preferred implementations, a logic module including functionality described herein delivers more accurate scoring than conventional EWS methodologies, reducing the number of false alerts and thus maintaining medical staff trust in the scoring.

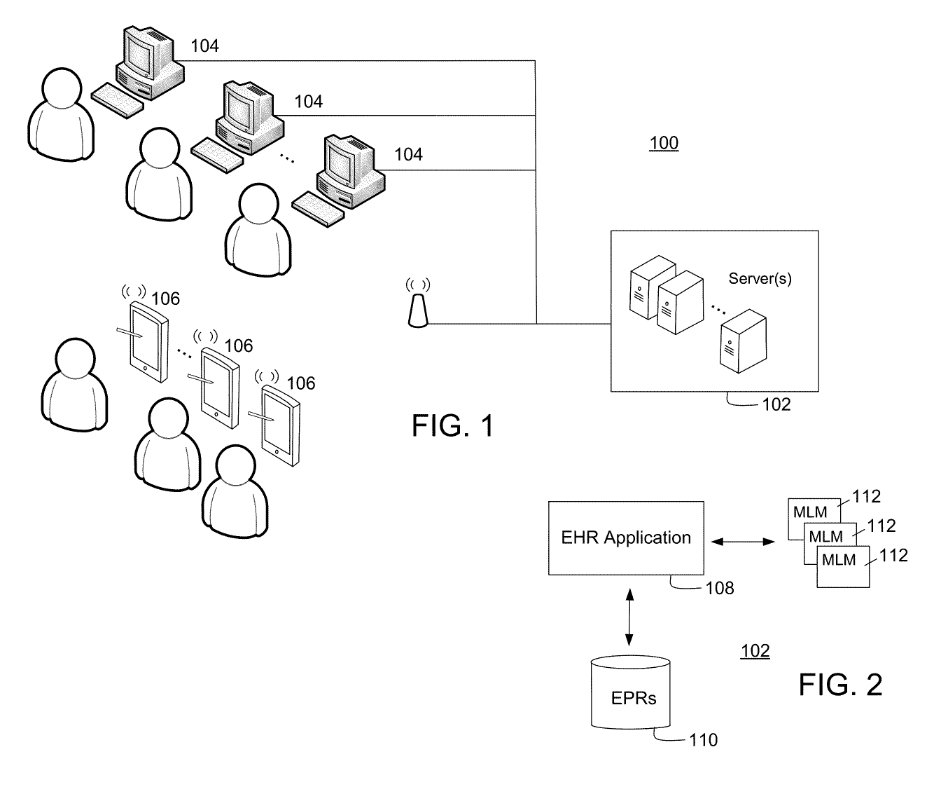

With reference now to the drawings, FIG. 1 schematically illustrates a computer network 100 in which one or more preferred embodiments of the present invention are incorporated. The computer network 100 is used in a medical context, inter alia, to calculate early warning scores for patients for use by hospital nursing and medical staff and emergency medical services. The calculated early warning scores are used to identify quickly the degree of illness or injury of a patient, and the early warning scores are based on data derived from physiological readings and other observations made by the medical personnel.

As illustrated, the computer network 100 includes a server system 102 comprising one or more computers. The server system 102 is arranged in electronic communication with one or more networked desktop computers 104 and one or more mobile computing devices 106 such as tablets and similar computing devices. The electronic communication can be wired, wireless, or a combination thereof.

FIG. 2 illustrates components of the server system 102 of the computer network 100 of FIG. 1. The server system includes an electronic health records (EHR) application 108, a database of electronic patient records (EPRs) 110, and one or more external logic modules 112.

The EHR application preferably is stored in non-transient computer readable medium of the server system 102, runs on one or more servers of the server system, and when run is accessible over the computer network by each of the computers and computing devices. Moreover, the EHR Application accesses the EPRs for storing and retrieving patient healthcare data to the respective records of the patients, and provides a means by which each of the computers and computing devices accesses the EPRs for reading and writing patient data. The EHR Application further accesses each of the logic modules, which are stored in non-transient computer readable medium of the server system.

Each logic module generally represents external computer-executable code that defines logic for performing one or more tasks by or for the EHR Application 108. For example, a logic module 112 may comprise a script for performance of a specific task for or on behalf of the EHR Application. In another example, a logic module may comprise an executable program for performing a specific task for or on behalf of the EHR Application. Furthermore, a logic module 112 is customizable, whereas the EHR Application itself generally is hardcoded with predefined logic and, although the EHR Application generally may be configurable to some extent upon installation and maintenance, the EHR Application generally is not customizable in terms of providing or modifying logic for performing specific tasks at each installation or location of use of an instance of the EHR Application. Moreover, a logic module can be attached to computerized flowsheets or called by dedicated software components outside of the EHR Application, allowing a range of applications to leverage the same logic module with consistency across those applications in the specific task performed by the logic module. The logic module can be easily maintained and modified without modifying the underlying EHR Application or modifying any other applications leveraging the logic module.

In accordance with one more preferred embodiments of the invention, early warning scores are calculated in a logic module, i.e., early warning scores are calculated in accordance with logic or computer-executable instructions stored in a logic module. By using a logic module to calculate the early warning scores, and by using the same underlying patient data from the EPRs, the logic for the calculations can be shared across a range of applications and components for providing consistency in the results of the calculations.

Due to the synergies and efficiencies provided by use of the logic module in calculating early warning scores, the logic of the logic module in one or more preferred embodiments is tailored to meet specific requirements of a facility or unit, e.g., to meet specific requirements of oncology, to meet specific requirements of oncology pediatrics, and to meet specific requirements of oncology emergency room. Such customization may be done through respective logic modules, with each corresponding to a facility or unit. Alternatively, a single logic module can be utilized that takes into account the specific requirements of the facility or unit based on an identification of the facility or unit.

For the same reasons, the logic of the logic module in one or more preferred embodiments is tailored to one or more specific patient groups, and the logic of the logic module in one or more preferred embodiments is tailored to one or more specific EWS standards as have been or may be established.

It will further be appreciated in view of the foregoing that EWS calculations can be tweaked or adjusted after implementation to fine tune utilization thereof in practice using try-inspect-adapt cycles or consensus methods.

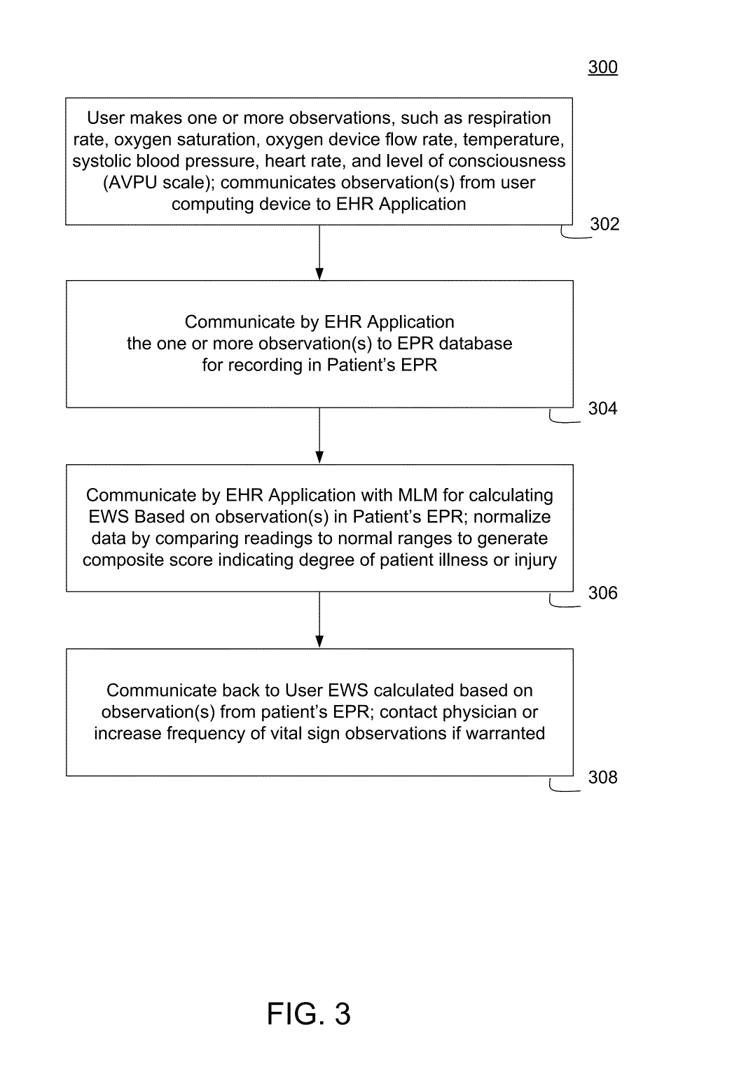

Turning now to FIG. 3, steps 302-308 of a broad method 300 of one or more preferred embodiments are illustrated. In accordance with step 302 of this method 300, observations are made for a patient. The observations may include respiration rate, oxygen saturation, oxygen device flow rate, temperature, systolic blood pressure, heart rate, and level of consciousness as determined, for example, using the AVPU scale. The one or more observations made by a user are communicated from a user device to the EHR Application.

In accordance with step 304 of this method 300, the one or more observations are communicated by the EHR Application to the EPR database for saving to the particular EPR for the patient. The data is thereby recorded and saved for future reference.

In accordance with step 306 of this method 300, the EHR Application communicates with a logic module, requesting a calculated early warning score for the particular patient, which is based on the recorded observations in the patient's EPR. Using the recorded observations, the logic of the logic module normalizes the data by comparing the readings to normal ranges to generate a composite score representing the degree of patient illness or injury.

In accordance with step 308 of this method 300, the EHR Application communicates with the user device and provides to the user the calculated early warning score for the patient based on the observations recorded in the EPR for the patient. In response to the calculated early warning score, and if the early warning score warrants such action, a physician may be contacted or the frequency of observations of vital signs of the patient may be increased.

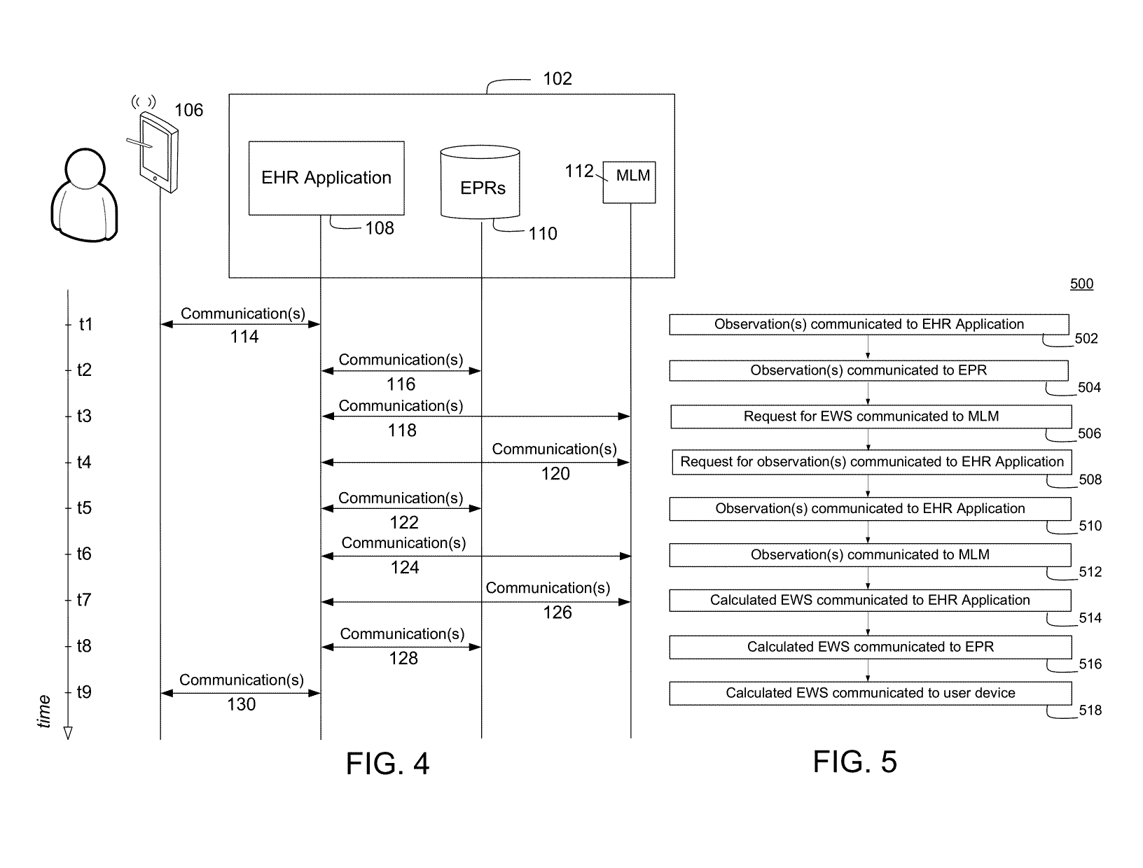

With regard to FIG. 4, a sequence of communications in one or more preferred embodiments of the invention are illustrated. Such communications occur between a user device comprising mobile computing device 106; an EHR Application 108; an EPR database 110; and a logic module 112. Furthermore, it will be appreciated that the user device of FIG. 4 is representative of computers and computing devices of FIG. 1; the EHR Application of FIG. 4 is representative of the EHR Application of FIG. 2; the EPR database of FIG. 4 is representative of the EPR database of FIG. 2; and the logic module of FIG. 4 is representative of each of the logic modules of FIG. 2.

In conjunction with FIG. 4, FIG. 5 illustrates steps 502-518 of a method 500 that in one or more preferred embodiments is performed through the sequence of communications illustrated in FIG. 4.

Referring now to FIGS. 4 and 5, in one or more first communications 114 at time t1, a device of a user communicates observations made regarding a patient to the EHR Application, which corresponds to step 502 in method 500. By reference here to “one or more communications”, and by such reference below, “one or more communications” is intended to include not only the sending of data from a first endpoint to a second endpoint in a computer network, but also any requests and responses that may be sent or received for establishing secure communications, as well as acknowledgement of receipt of a message, all of which support the sending of the data from the first endpoint to the second endpoint in the computer network.

In one or more second communications 116 at time t2, the EHR Application communicates the observations to the EPR database for recording in the EPR of the specific patient for which the observations were made by the user, which corresponds to step 504 in method 500.

In one or more third communications 118 at time t3, the EHR Application communicates with a particular logic module for calculating an early warning score for the patient. Such communication may include a request for calculation of an early warning score, including an identification of the patient for which the score is to be calculated in accordance with the logic module's logic. This corresponds to step 506 in method 500.

In one or more fourth communications 120 at time t4, the logic module communicates with the EHR Application requesting one or more observations for use in calculating the early warning score requested for the patient. Such communication may include a request for specific observations and other patient data from the patient's EPR that are used in calculation of the early warning score, per the logic module's logic, and an identification of the patient for which the score is to be calculated in accordance with the logic module's logic. This corresponds to step 508 in method 500.

In one or more fifth communications 122 at time t5, the EHR Application communicates with the EPR database for retrieving the one or more observations and other patient data from the patient's EPR, as required by the logic module. The data is read from the patient's EPR and returned to the EHR Application. This corresponds to step 510 in method 500.

In one or more sixth communications 124 at time t6, the EHR Application communicates with the particular logic module, communicating the one or more observations and other patient data retrieved from the patient's EPR for calculating the early warning score for the patient. This corresponds to step 512 in method 500.

In one or more seventh communications 126 at time t7, the logic module communicates the calculated early warning score for the patient based on the retrieved one or more observations and patient data. The early warning score is communicated to the EHR Application. This corresponds to step 514 in method 500.

In one or more eighth communications 128 at time t8, the EHR Application communicates the calculated early warning score to the EPR database for recording in the EPR of the specific patient for which the score was calculated, which corresponds to step 516 in method 500.

In one or more ninth communications 130 at time t9, the EHR Application communicates with the user device the calculated early warning score, which is then displayed to the user on the user's device. This corresponds to step 518 in method 500.

It will be appreciated that while the foregoing descriptions related to FIGS. 4-5 have described and treated logic modules as executable software separate from and able to communicate with the EHR Application, each logic module may be a script or code that is executed by the EHR Application itself. Even in such scenarios, each logic module remains a module that is customizable independently of the EHR Application and its underlying source code. Thus, as between different facilities of different healthcare systems, the EHR Application may be the same but the logic modules may differ, resulting in different methodologies in the calculations of the early warning scores.

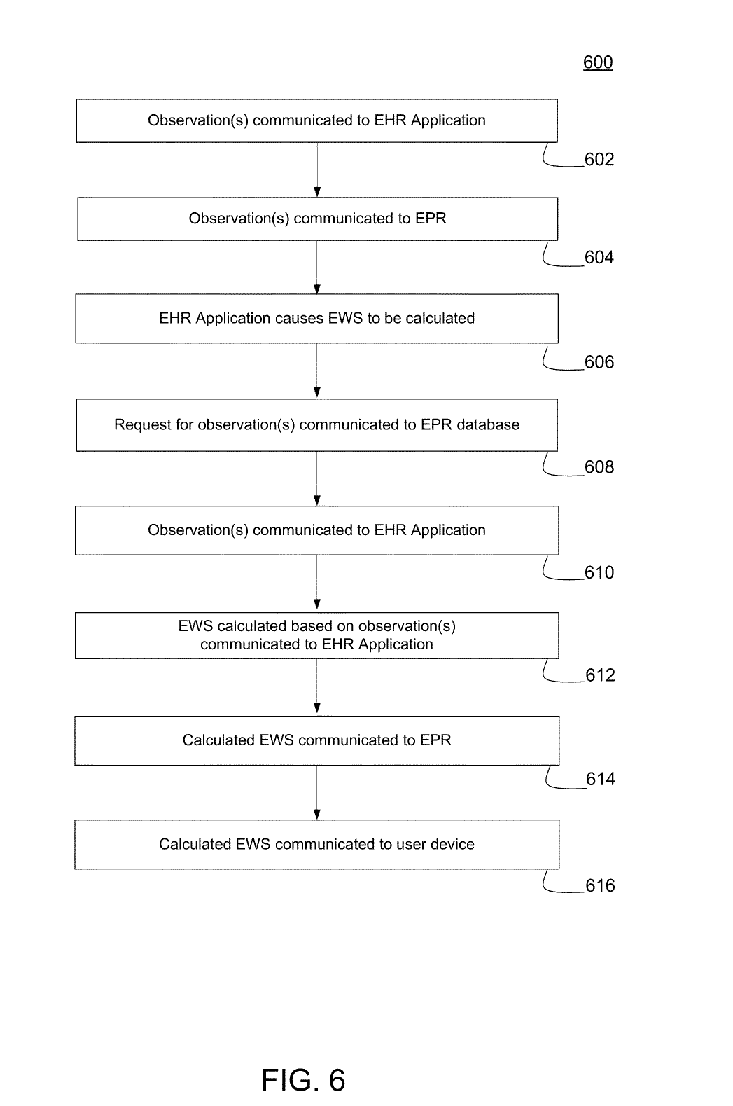

FIG. 6 is similar to the method illustrated in FIG. 5, with the difference being that the logic module represents a script that is executed by the EHR Application and, thus, the communications shown between the EHR Application and the logic module in method 500 have been omitted in the method 600 of FIG. 6.

In particular, FIG. 6 illustrates steps 602-616 of another method 600 that, in one or more preferred embodiments, is performed through a sequence of communications. In this regard, one or more observation(s) made by a user are communicated from a device of the user to the EHR Application at step 602; the observations are then communicated to the EPR database for recording in the applicable patient EPR at step 604; at step 606 a request for an early warning score is received by the EHR Application or, alternatively, an event occurs that triggers the EHR Application to cause an early warning score to be calculated for a patient (such event being, for example, the recording of one or more observations to the EPR for such patient); at step 608 a request for one or more observations as recorded in the patient's EPR is communicated to the EPR database; at step 610 the requested one or more observations are communicated to the EHR Application; at step 612 the early warning score for the patient is calculated based on the observations retrieved from the patient's EPR; at step 614 the calculated early warning score is communicated to the EPR database for recording in the patient's EPR; and at step 616 the calculated early warning score is communicated to a user device for display to a user.

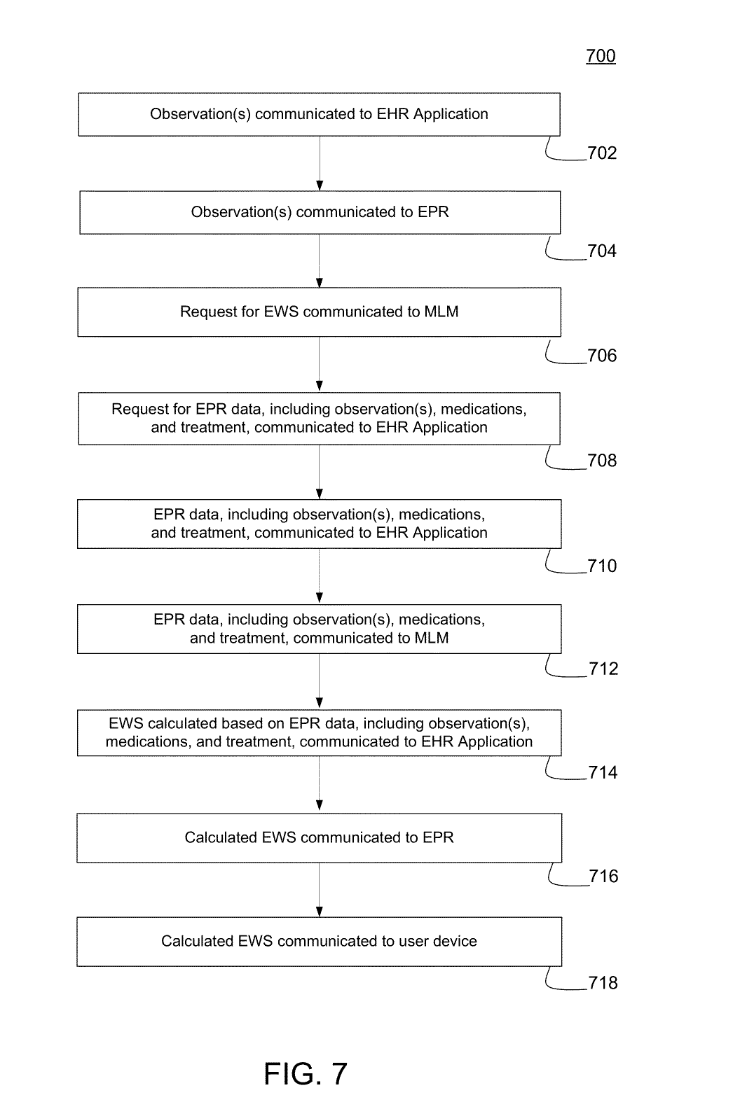

FIG. 7 illustrates steps 702-718 of another method 700 that is performed in one or more preferred embodiments through the sequence of communications illustrated in FIG. 4. In FIG. 7, the early warning score is calculated based not only on observations, but also on medication and treatment information recorded in the patient's EPR. Calculation of the early warning score thus takes into account, for example, a patient receiving oxygen therapy, for which the system may lower what is considered to be a normal blood oxygen saturation range for such patient.

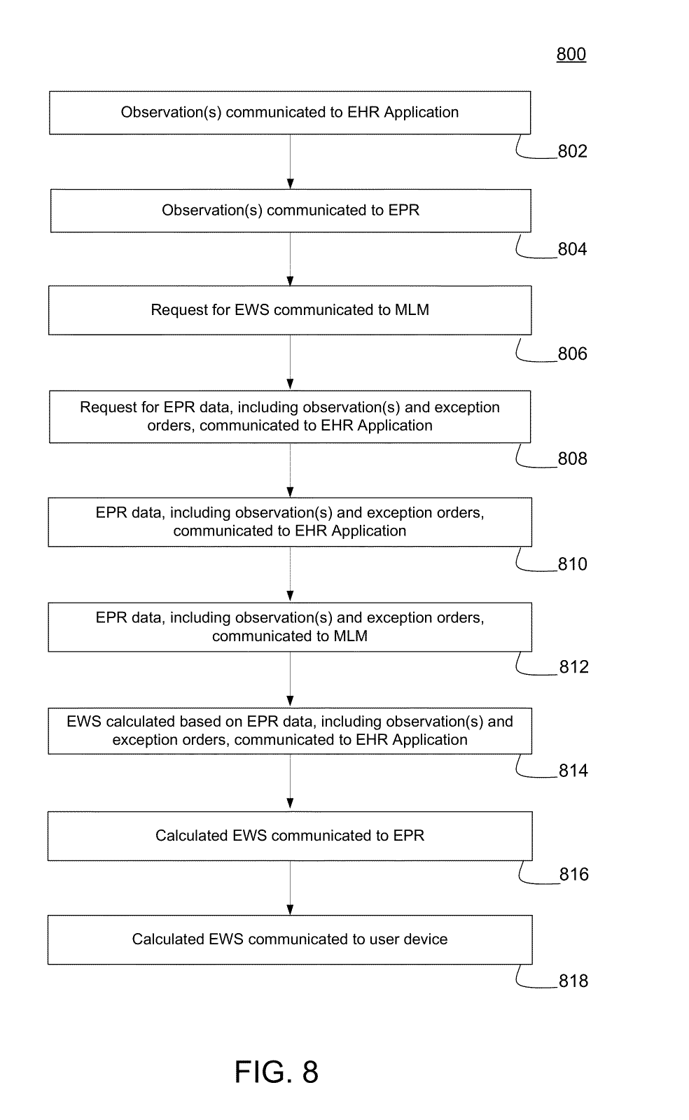

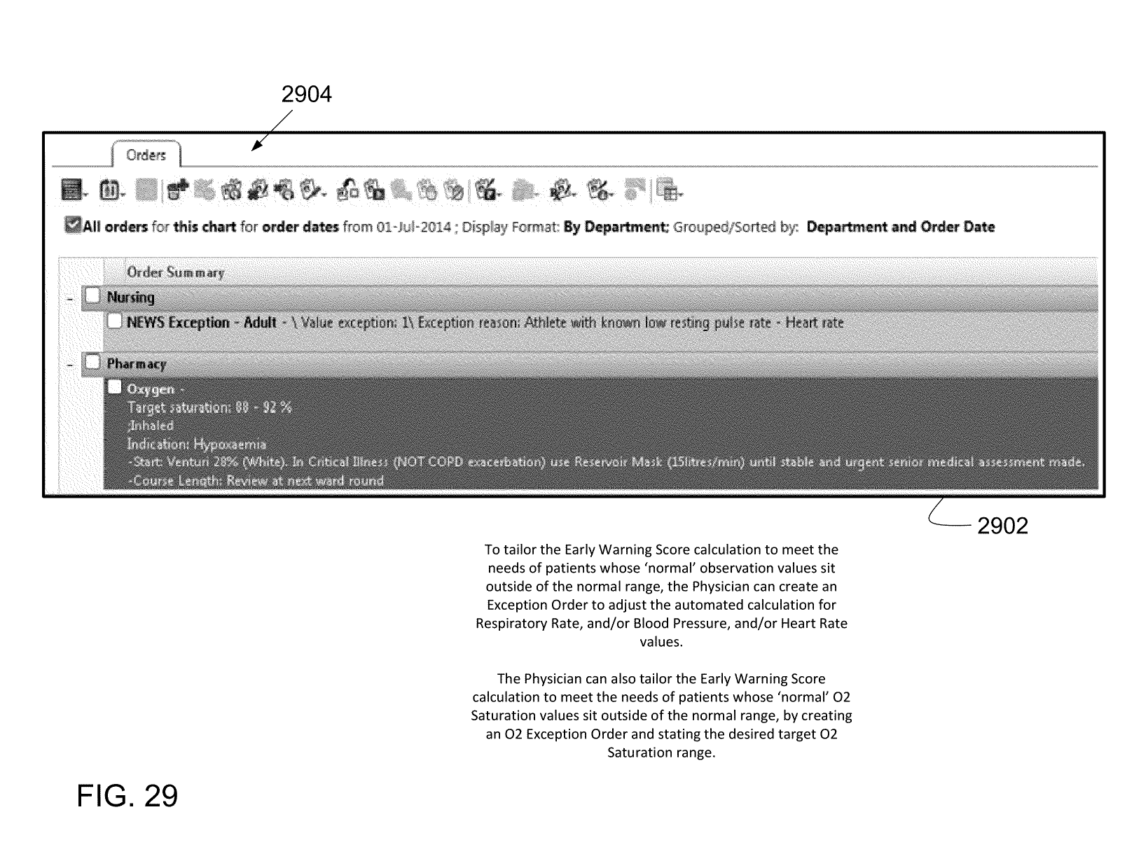

FIG. 8 illustrates steps 802-788 of another method 800 that is performed in one or more preferred embodiments through the sequence of communications illustrated in FIG. 4. In FIG. 8, the early warning score is calculated based not only on observations, but also on exception orders recorded in the patient's EPR. An example of an exception order as revealed in an EHR Application and recorded in the patient's EPR is shown in FIG. 29. Calculation of the early warning score thus takes into account, for example, orders to offset the scoring of individual observations such as oxygen therapy order minimum and maximum oxygen saturation; heart rate exception orders; respiration rate exception orders; and blood pressure orders.

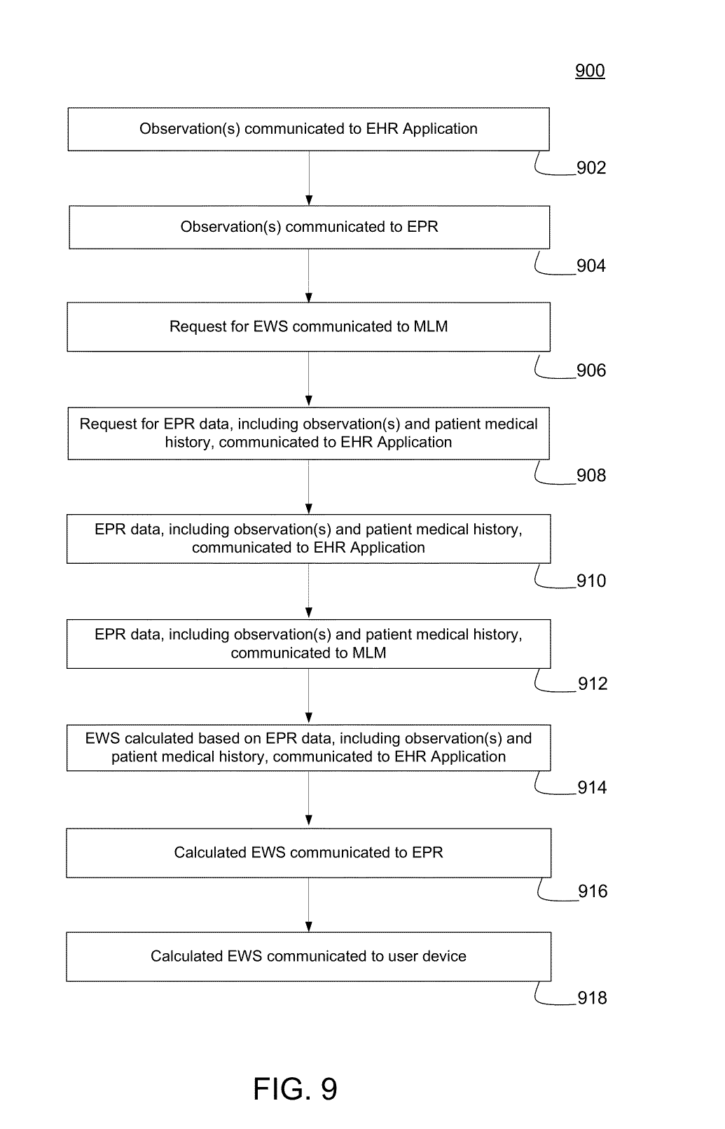

FIG. 9 illustrates steps 902-918 of another method 900 that is performed in one or more preferred embodiments through the sequence of communications illustrated in FIG. 4. In FIG. 9, the early warning score is calculated based not only on observations, but also on the patient's medical history recorded in the patient's EPR. Calculation of the early warning score thus takes into account, for example, a known history of high blood pressure.

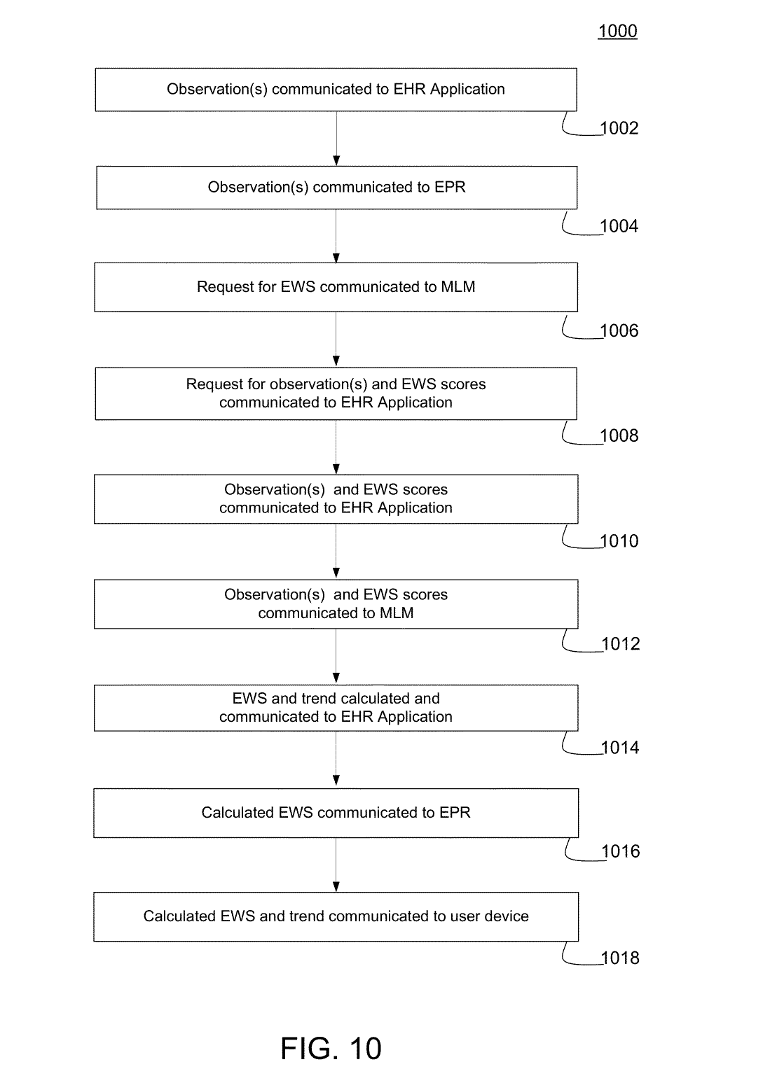

FIG. 10 illustrates steps 1002-1018 of another method 1000 that is performed in one or more preferred embodiments through the sequence of communications illustrated in FIG. 4. In FIG. 10, the early warning score is calculated together with a trend of the early warning score based on early warning scores that were recorded in the patient's EPR. Thus, both an early warning score and an indication of whether such score is trending higher or lower is provided to the user device for display to a user. Early warning scores are thereby tracked over time, allowing medical staff to view not just a patient's current score, but also whether the patient's score is increasing (e.g. patient is deteriorating) or decreasing (e.g. patient is improving) over a period of time.

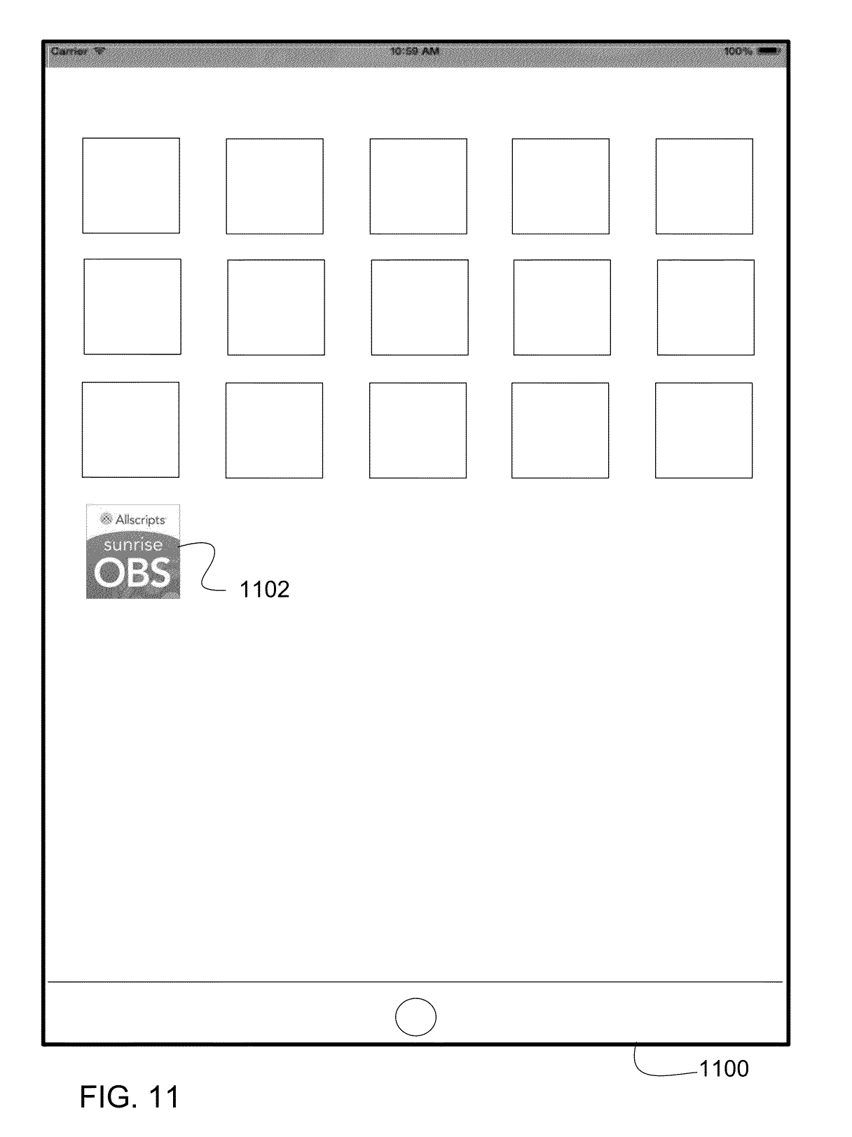

FIGS. 11-28 illustrate an application that is run on an iOS mobile device 1100 through which observations are made and early warning scores are viewed by a user, such as a nurse. The iOS mobile device 1100 is representative of the computers and computing devices of FIG. 1 and the illustrated use of the iOS mobile device 1100 in FIGS. 11-28 is within the context of the computer network 100 of FIG. 1 and server system 102 of FIGS. 1 and 2.

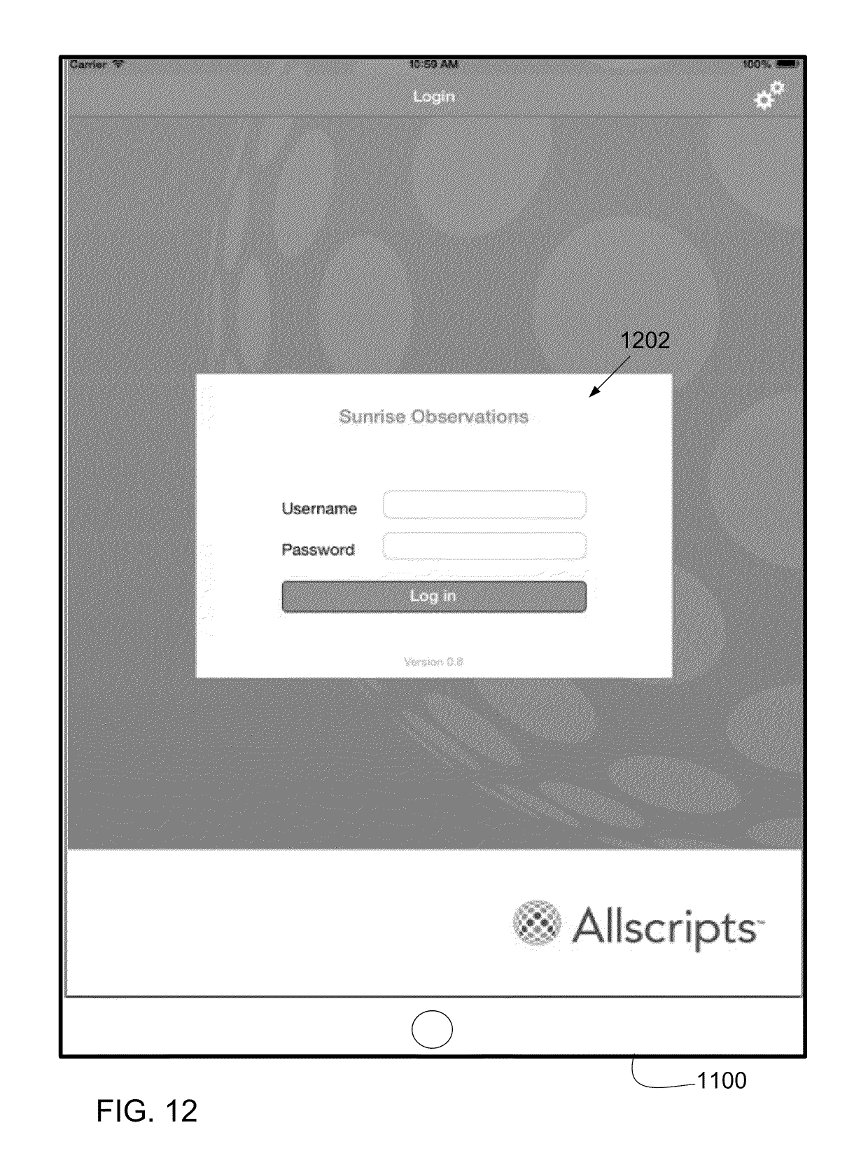

In FIG. 11, a home screen of the iOS mobile device 1100 is shown with icons representing apps arranged thereon. Icon 1102 represents the “observations” app that works with Sunrise Clinical Manager by Allscripts, as mentioned above. Selecting icon 1102 displays a login box 1202 for authentication of the user, as shown in FIG. 12.

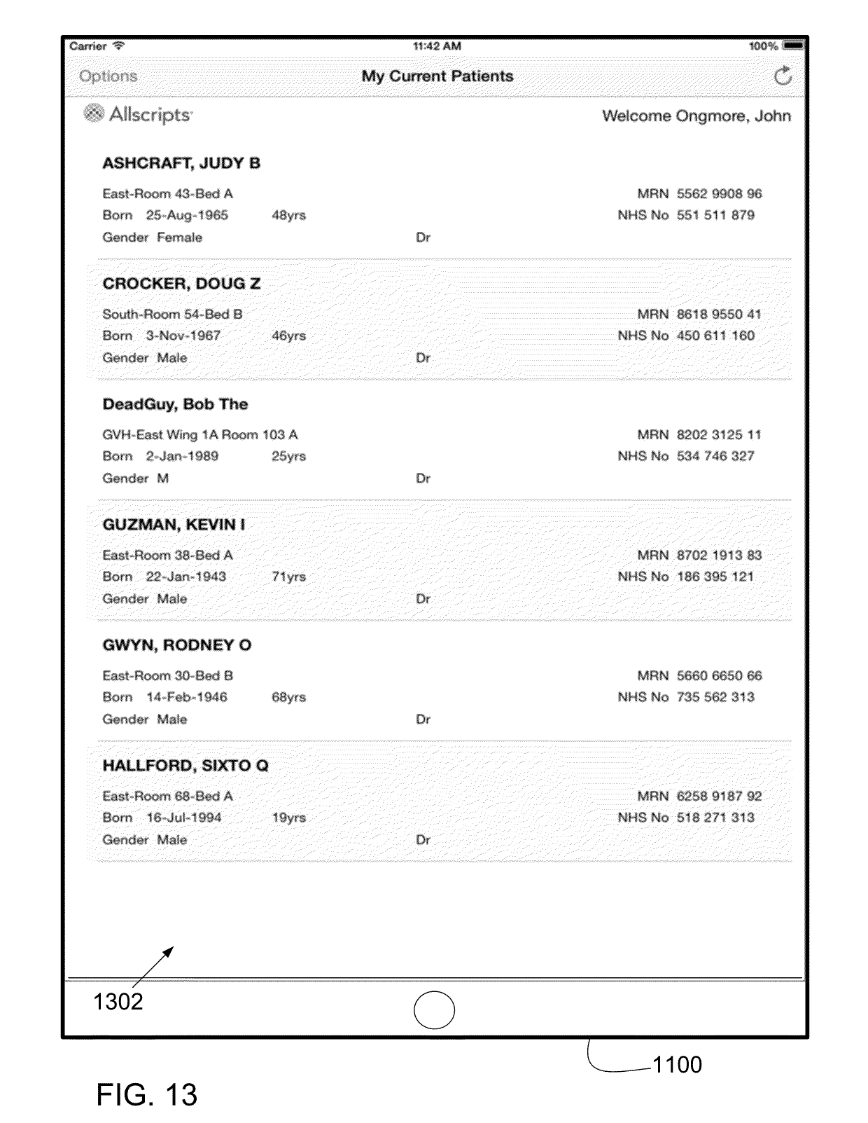

Following a successful authentication, a user interface 1302 for listing patients for whom observations can be made is made accessible to the user and is displayed in FIG. 13, wherein a representative listing of patients is shown. Preferably, the listing is the same “Patient List” that is configured as the user's default “Patient List” in Sunrise Clinical Manager, i.e., the EHR Application.

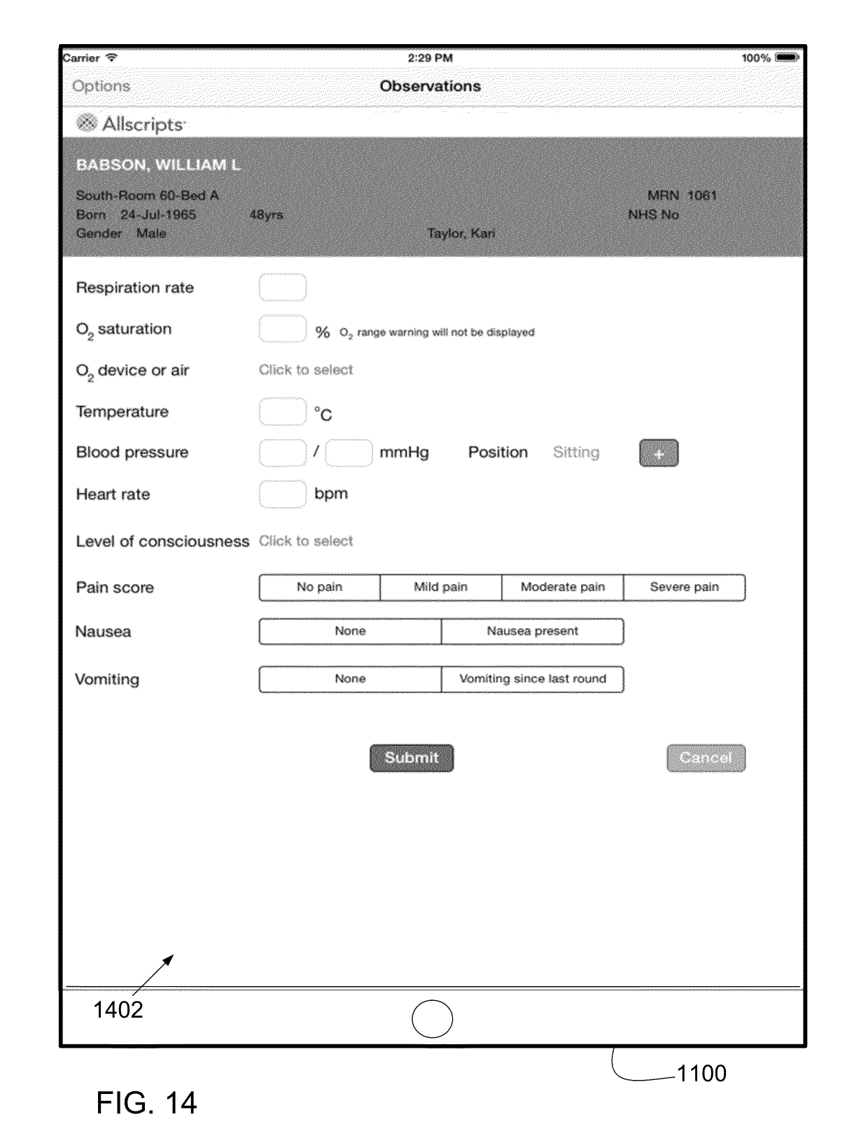

FIG. 14 illustrates a user interface 1402 for receiving user input regarding observations made by a user regarding a specific patient. The user interface 1402 is presented upon selecting a patient from the patient listing as represented in FIG. 13. Upon selection of a patient, the user interface 1402 is displayed with the selected patient in context.

With further regard to the preferred user interface 1402 that is displayed, the specific patient is identified at the top of the user interface 1402 in a patient banner. Elements of the user interface for receiving user input are provided there below. As shown, user interface 1402 includes user input elements for receiving user input representing observations made with respect to the patient's respiration rate; the patient's oxygen saturation; whether the patient has an oxygen device or is breathing ambient air; the patient's temperature; the patient's blood pressure; the patient's heart rate; the patient's level of consciousness; the patient's pain level; whether the patient is experience nausea; and whether the patient has experienced vomiting.

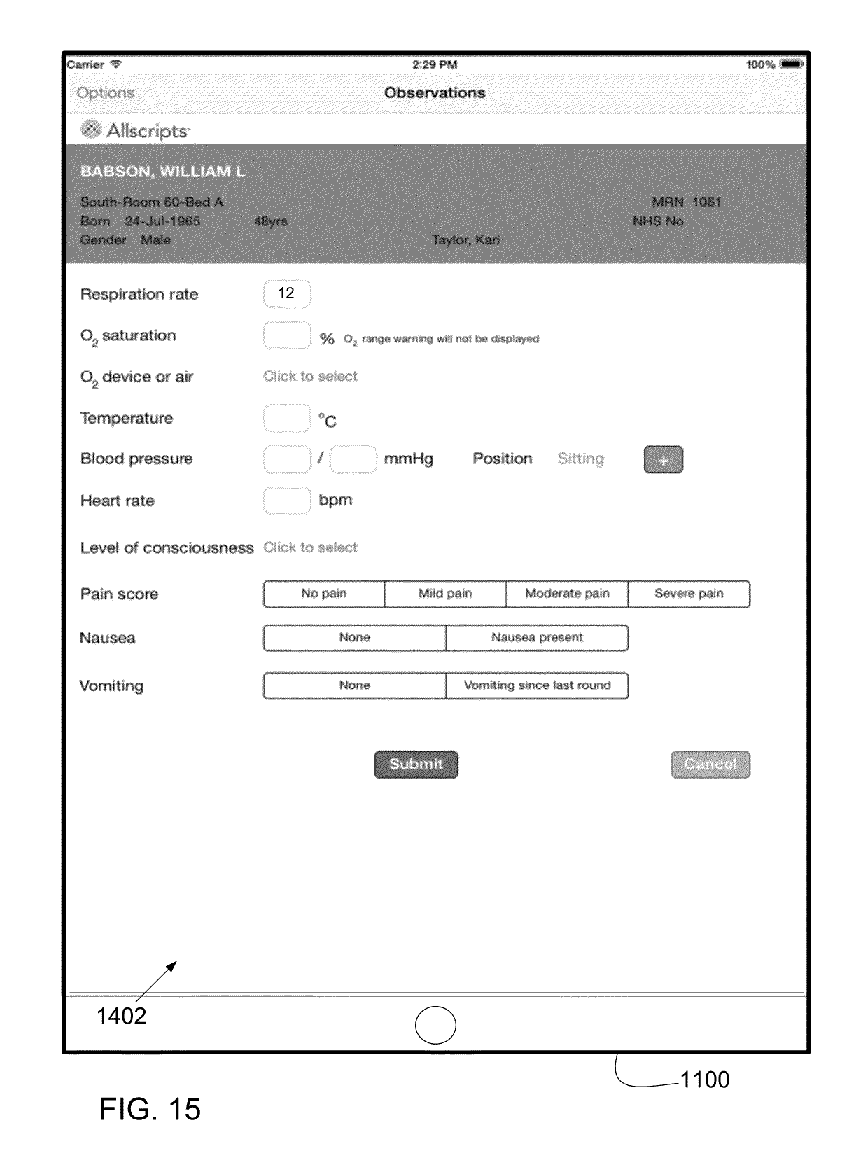

FIG. 15 illustrates user input being received from the user regarding the patient's respiration rate. In this example, the user input field for respiration rate is selected by the user by touching the area of the display of the iOS device 1100 corresponding to that user input field. This enables the user then to input a value for the respiration rate of the patient. As shown in FIG. 15, the value of “12” has been entered by a user for the patient's respiration rate. If thereafter a user desires to change the value that has been entered, the user can select the input field again and change the value. This can be done until such time as the observations are successfully submitted by selecting the “submit” button of the user interface 1402.

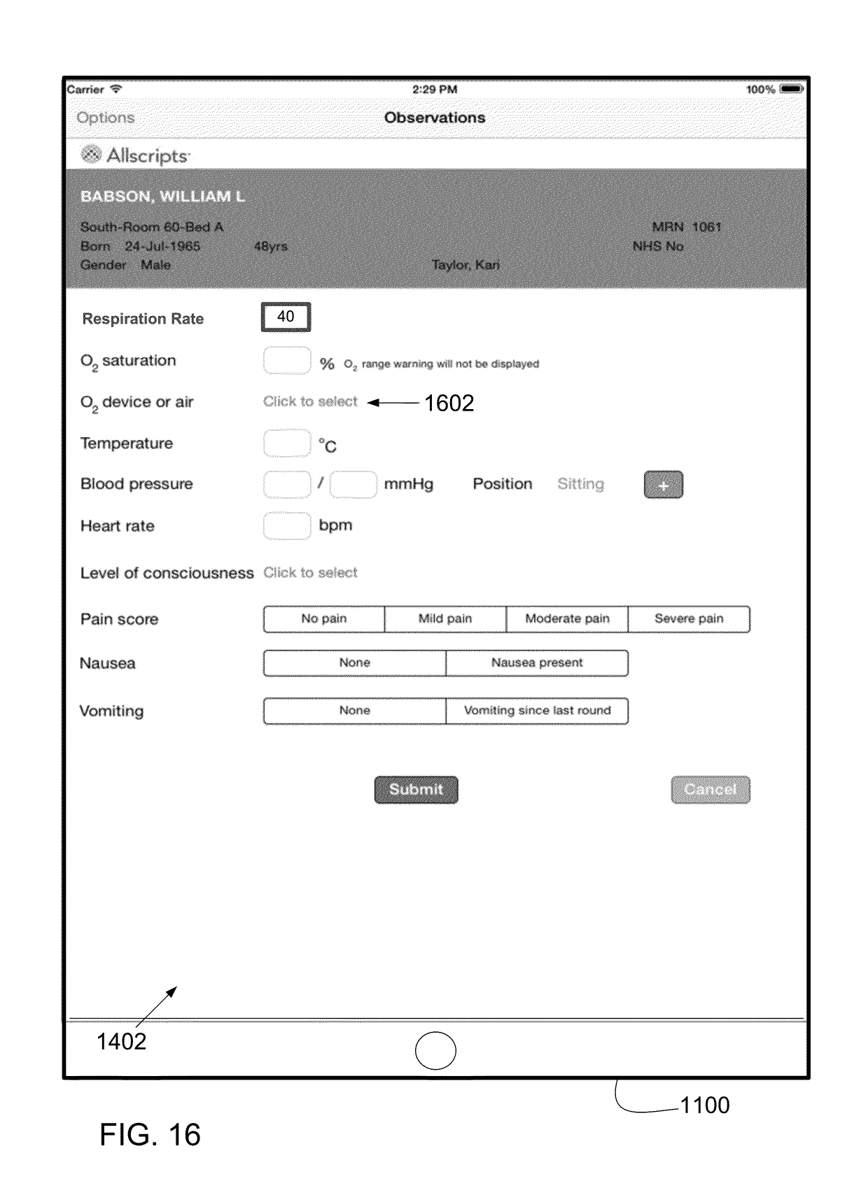

FIG. 16 illustrates user input of a value for the respiration rate that is outside of a normal range, wherein feedback is provided by the application by changing the field name font to red, bolding the font, and highlighting the user input field with a thick red line. (As used here, a “normal range” for respiration rate refers to an observation value that scores 0 in the Salford Royal Foundation Trust Adult Observation Chart, 6th draft, Oct. 8, 2013). For a field for which such feedback is not provided, the user is informed of this in the user interface 1402. For example, for the oxygen saturation note field, the user is notified that feedback will not be provided when the value entered is outside of normal limits due to varying target oxygen ranges.

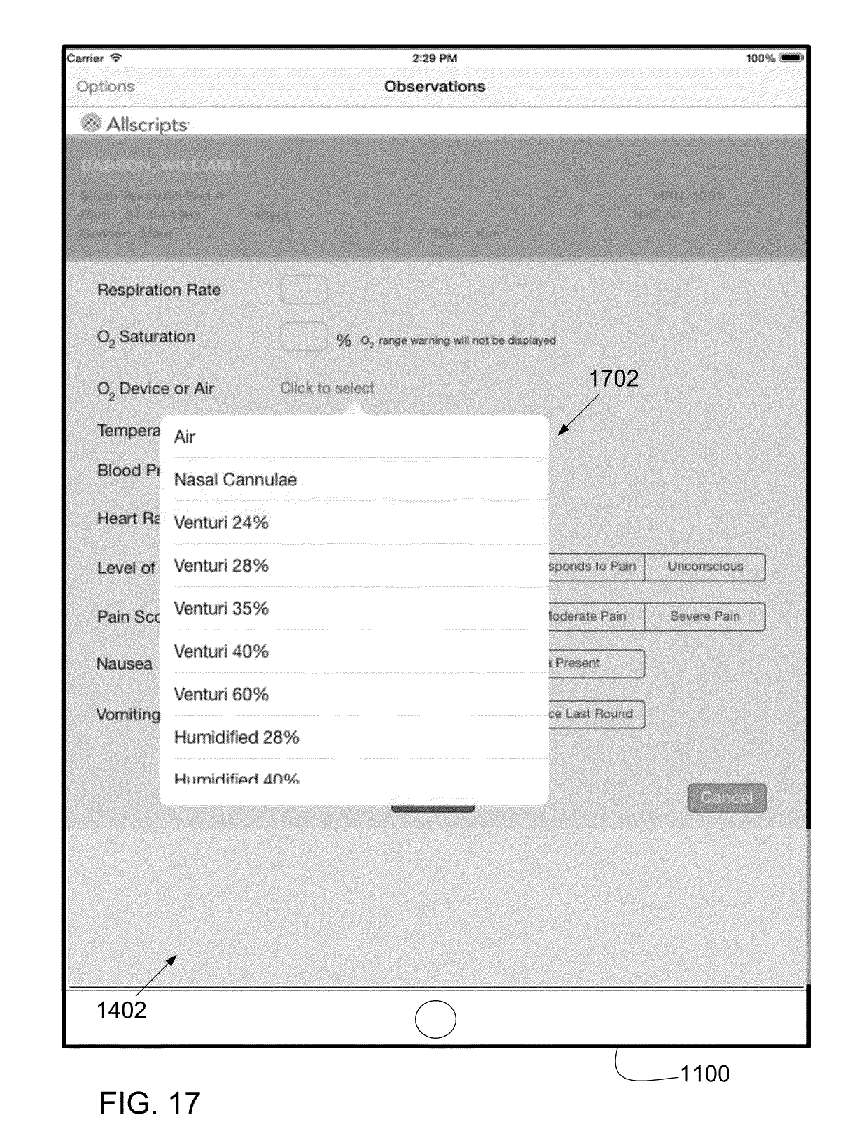

FIG. 17 illustrates a secondary user interface 1702 that appears when the “Click to select” element 1602 of the user interface 1402 shown in FIG. 17 is selected. This element 1602 preferably is selected when the patient is receiving oxygen via an oxygen device. While this secondary user interface 1702 is presented, the underlying user interface 1402 is “grayed out” and rendered inaccessible for input by the user. The secondary user interface 1702 provides a list of oxygen devices and configurations for selection of one by the user.

As shown in FIG. 17, the options for selection include “Nasal Cannulae”, “Venturi 24%”, “Venturi 28%”, “Venturi 35%”, “Venturi 40%”, Venturi 60%”, “Humidified 28%”, and “Humidified 40%”. Additional options are presented outside of the current view shown, and are accessible by scrolling down to view those selections. Preferably the last option for selection is “Other”, the selection of which results in a text box allowing the user to enter the type of oxygen device being used. Furthermore, the first option preferably is “Air”, which is selected if no oxygen device is being used to confirm that no device in fact is being used.

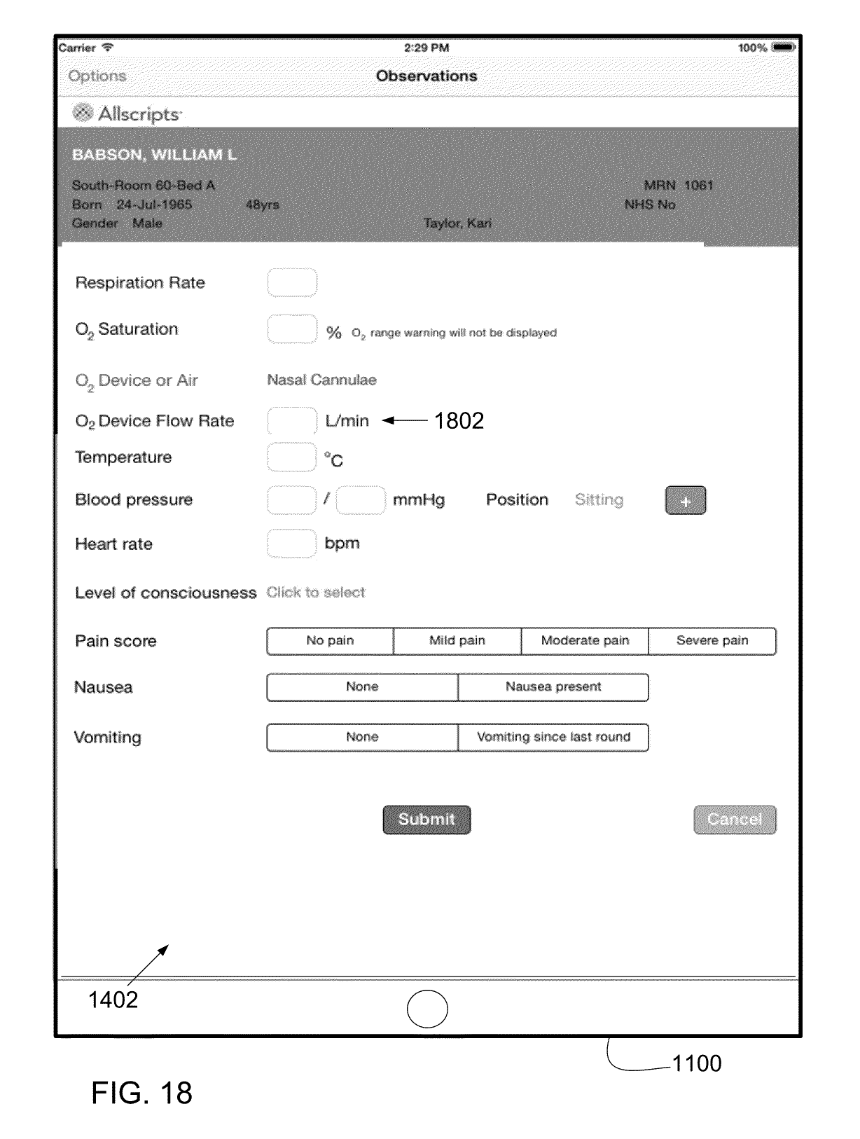

When an oxygen device is selected, then the option selected will appear in place of “Click to select” shown in FIG. 17. Indeed, “Nasal Cannulae” has been selected in the secondary user interface 1702 and, consequently, “Nasal Cannulae” is displayed in the user interface 1402 as seen in FIG. 18. Furthermore, since confirmation by the user that an oxygen device is being used has been received, a new user input field 1802 is displayed in user interface 1402 regarding the device selected. The new user input field that is displayed is used to input the device flow rate in units of liters per minute.

Furthermore, as shown in FIG. 18, the “02 Device or Air” field label text, and the text showing the selection in secondary user interface 1702—is red, and the text showing the option selected additionally is bold, when an oxygen device is selected and the new user input field for entering the oxygen device flow rate is displayed. As will be appreciated, other user input fields similarly may be displayed, or may cease to be displayed, as a function of user input that is received via user interface 1402.

Once the desired observations have been made by the user, the user selects the “Submit” button to record those observations in the electronic patient record (EPR) for the identified patient. Following submission, the user interface 1402 preferably resets, whereby the same observations that were submitted are not carried forward automatically in a subsequent submission. Furthermore, one or more observations may be submitted. For example, if some observations already were submitted at the same time by the user, then observations supplementing those may be made immediately following such submission wherein just the supplemental observations are made in user interface 1402. Preferably there is not requirement that all user input fields be completed by a user before submitting one or more observations for recording in the patient's EPR.

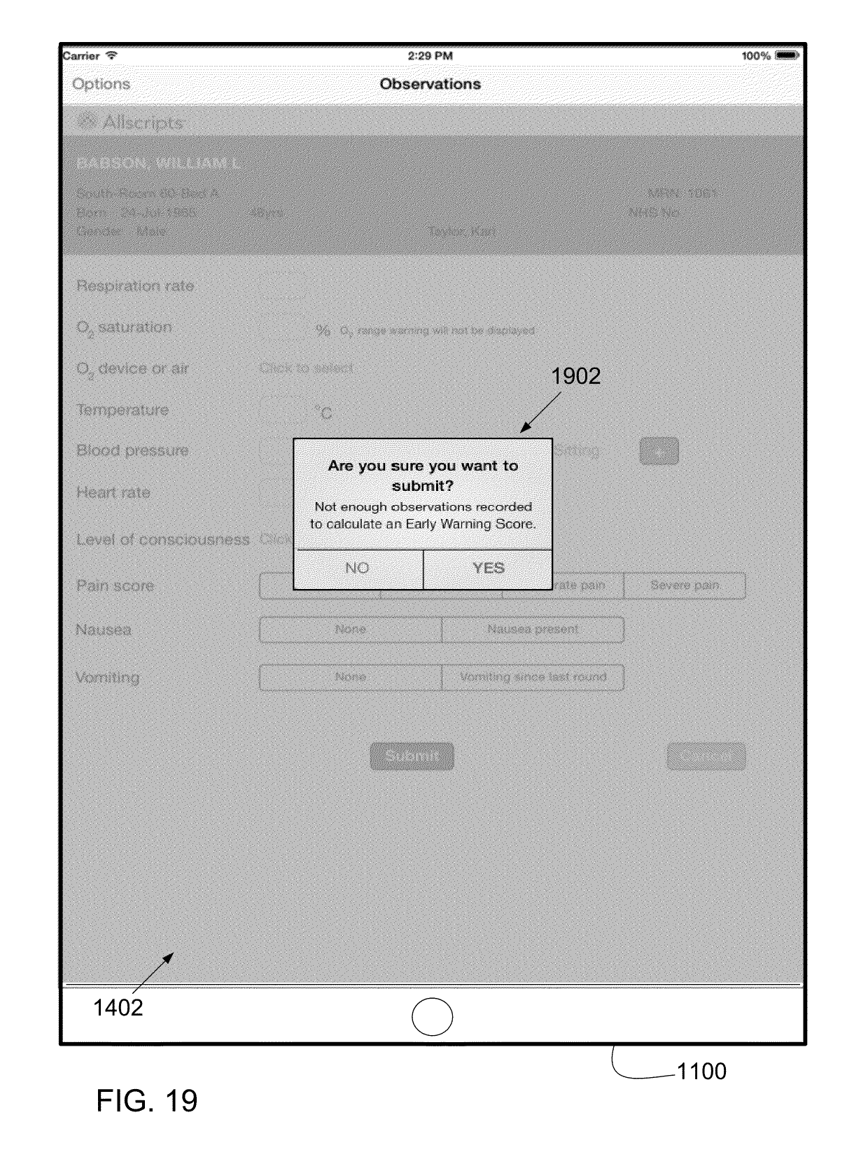

If the submit button is selected, and if there is an insufficient number of observations being submitted so as to calculate an early warning score based thereon, then a user interface box 1902 preferably is displayed alerting the user that there are insufficient observations being submitted to calculate an early warning score based thereon. The user is presented through user interface box 1902 the choice of making the submission or returning to the user interface 1402 for making additional observations. This is illustrated in FIG. 19.

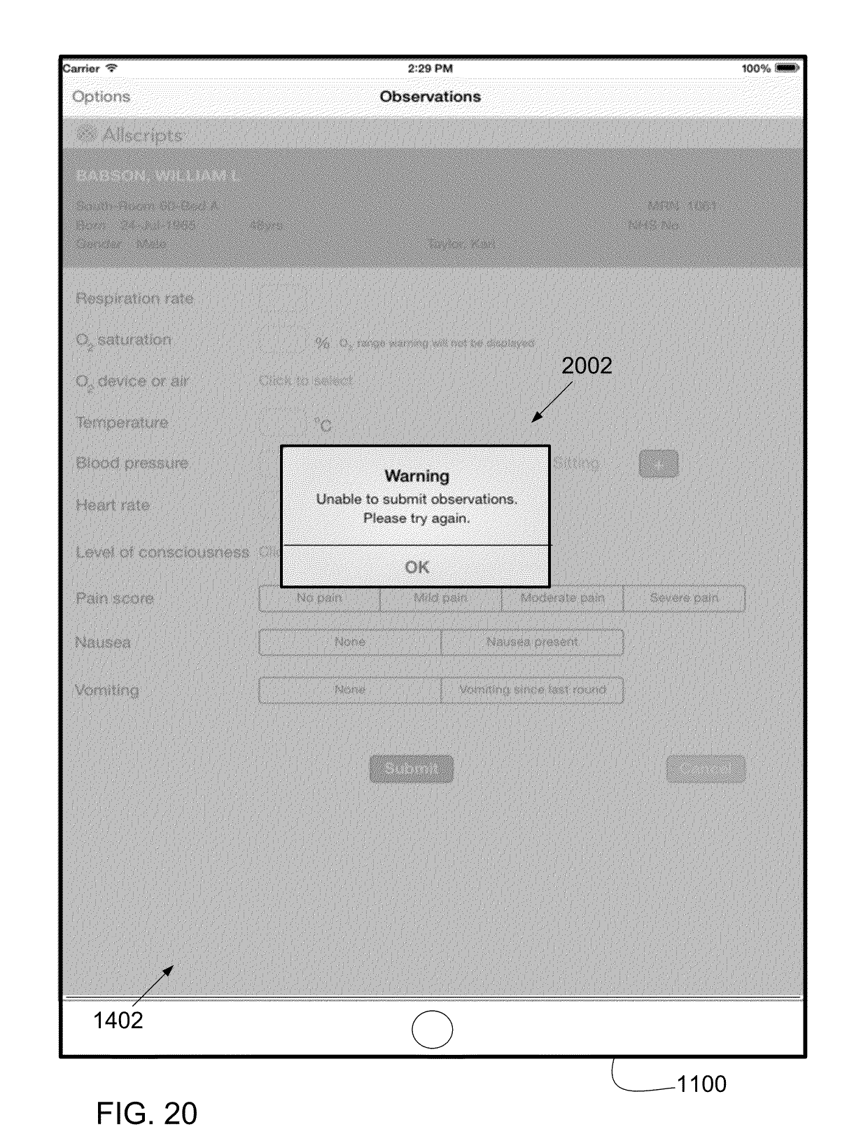

As will be appreciated, the iOS mobile device 1100 communicates with the computer network via a wireless access point (which is schematically represented at 111 in FIG. 1). If a connection is lost between the device 1100 and the network and the submission is unable to be made, then the user preferably is alerted as shown, for example, in FIG. 20, via a user interface box 2002. Upon acknowledgement by the user, indicated by selecting “OK”, the user interface 1402 is displayed with the observations of the attempted submission. The user can wait a few moments and try the submission again.

If the user instead turns off the iOS mobile device 1100 or exits the observations application, the observations made preferably are lost. Indeed, the user input data preferably is not stored on the iOS mobile device 1100 other than in connection with being cached in transient memory by virtue of a value received in the user input fields of the user interface 1402. This configuration is intended to prevent any protected healthcare information from being stored in non-transitory computer readable medium of the iOS mobile device 1100. Essentially, the iOS mobile device 1100 serves as an interface to the EHR Application by the user. Moreover, in this respect, it will be appreciated that the patient listing, and other protected healthcare information, is loaded into the program via the computer network when the observations application is launched, and such information also is not stored in non-transitory computer readable medium of the iOS mobile device 1100.

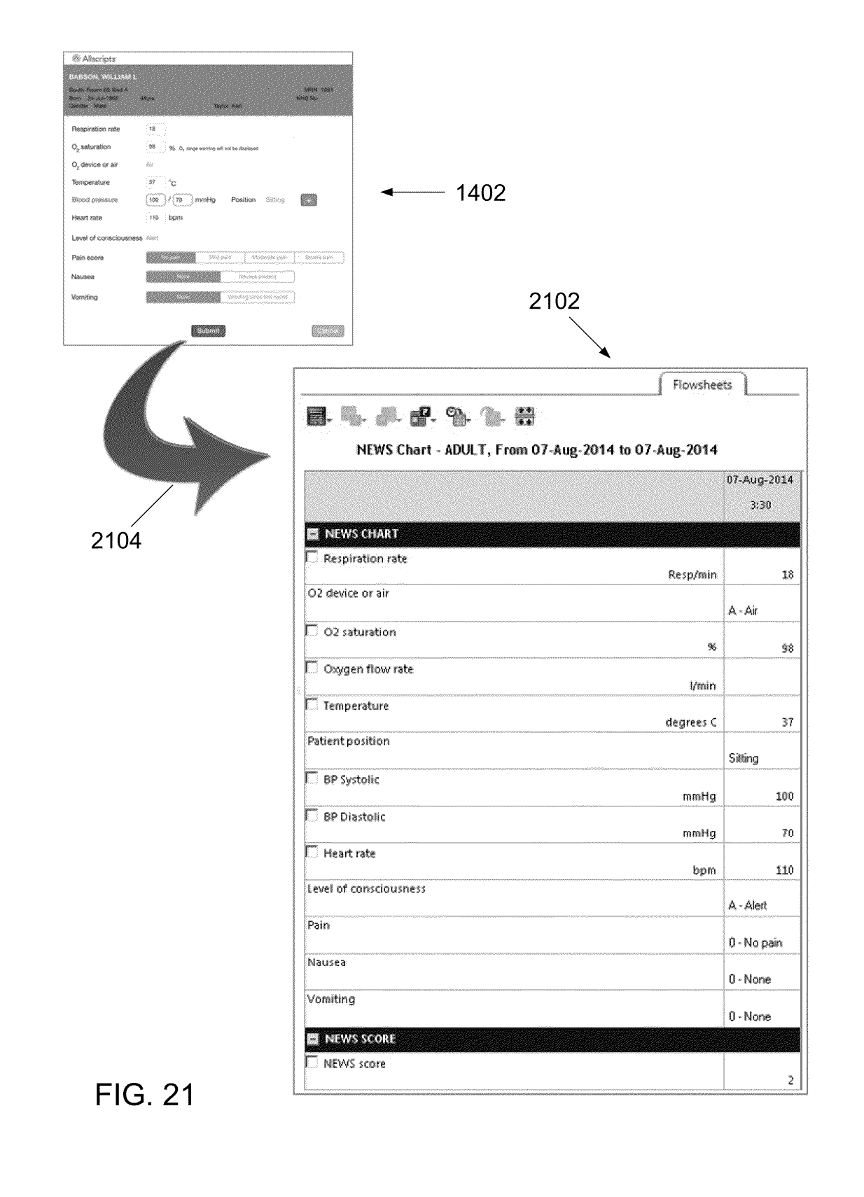

FIG. 21 illustrates the transfer of submitted observations from the iOS mobile device 1100 to a “flowsheets” tabbed page within an EHR Application, which page is in the context of the identified patient and which data forms part of the patient electronic record of the patient. It will be seen from FIG. 21 that the observations made as seen in the user interface 1402 in FIG. 21 are reflected—after submission, which act of submission is represented by the arrow 2104 in FIG. 21—in the displayed tabbed page 2102 in FIG. 21. It will further be appreciated that the user, e.g., a nurse, is able to manually insert an observation value directly into the flowsheet and also receive the same calculated early warning score within the EHR application, as shown.

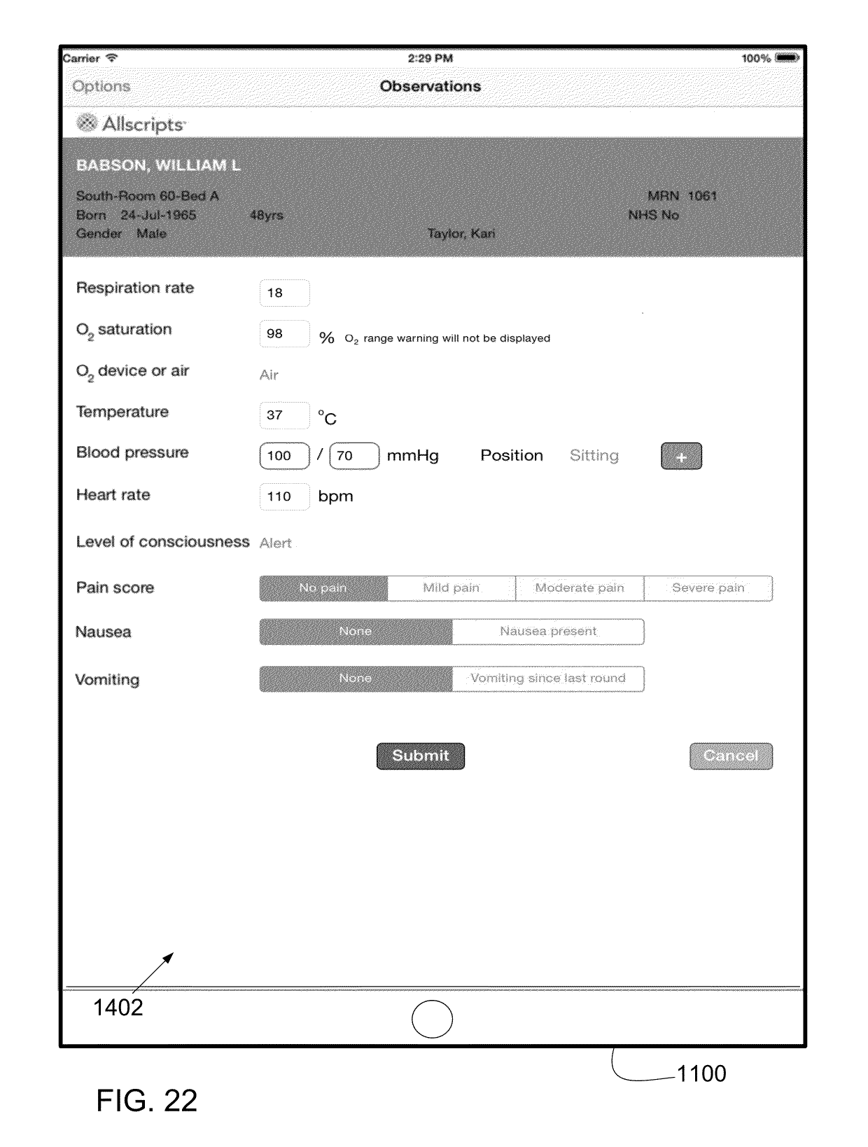

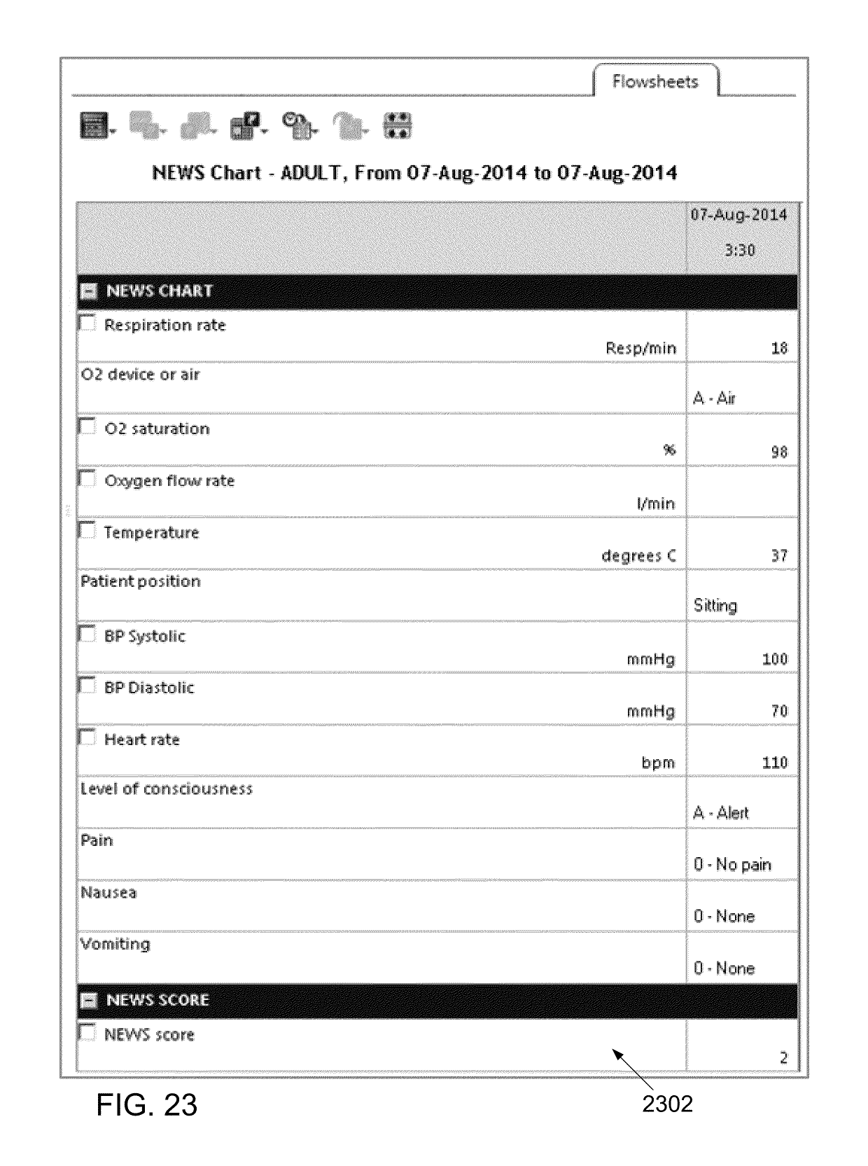

For clarity, the user interface 1402 of FIG. 21 is reproduced in FIG. 22 as would be seen on iOS mobile device 1100, and the tabbed page 2102 is reproduced in FIG. 23. Based on the observations made, the tabbed page 2102 shows the calculated early warning score—here a “2”, which has been calculated in accordance with one or more preferred embodiments of the invention.

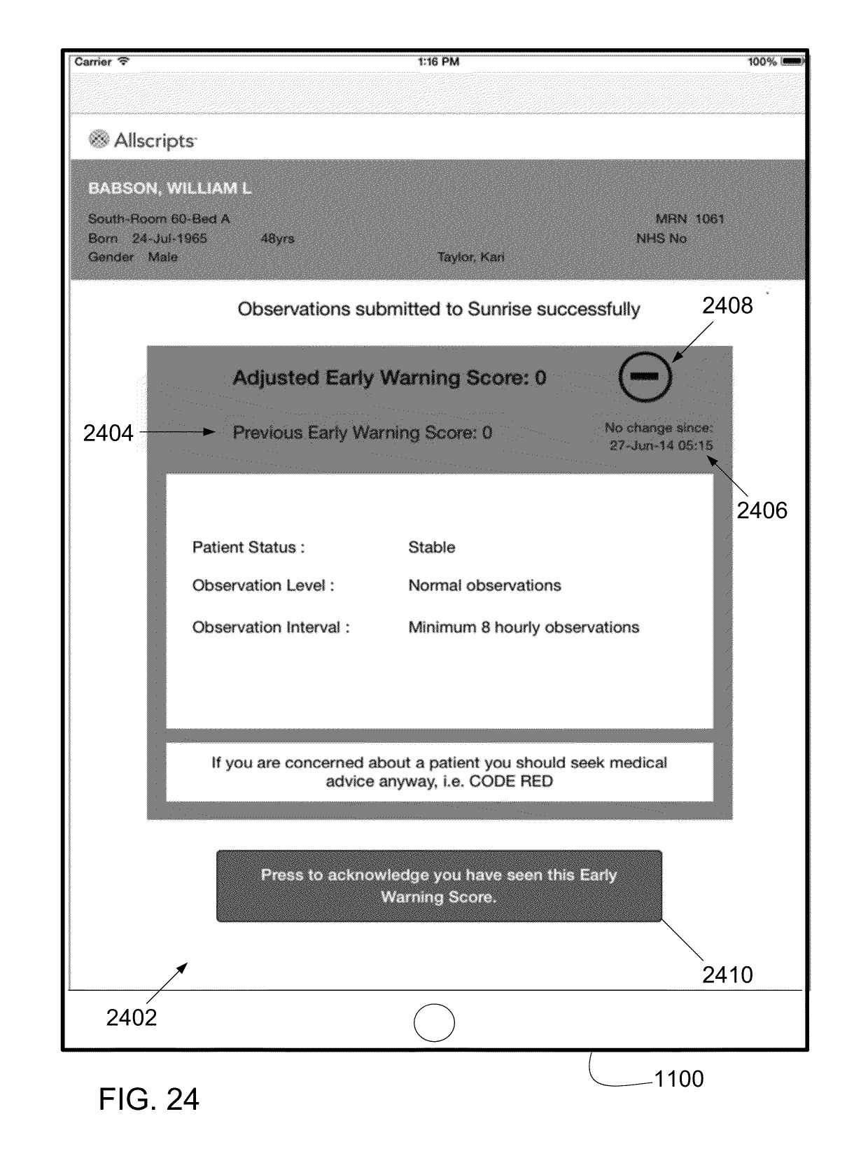

It will further be appreciated that it would be beneficial to display the calculated early warning score that incorporates the most recent submissions to the user of iOS mobile device 1100 upon submitting observations. In this respect, FIGS. 24-28 illustrate different user interfaces for displaying a calculated early warning score and alerting the user regarding the same, if warranted.

Thus, for example, FIG. 24 illustrates the display to a user on iOS mobile device 1100 of a user interface 2402 for an early warning score calculated following submission, wherein the score is calculated to be a “0”. This indicates that the patient's status is “Stable”; that the observations are normal; and that observations should be made at intervals of a minimum of eight hours. The user interface 2402 includes a “green” theme indicating the patient status and favorable early warning score that has been calculated. A similar user interface is displayed for calculated scores of 1 and 2.

If an early warning score has been previously calculated, as recorded in the EPR for the patient, then the user interface 2402 further displays the early warning score at 2404, which here was previously calculated to be “0”; further displays that the early warning score is without change at 2406; and further displays a visual indication of no change in the score at 2408; as shown here the “dash” in the circle indicates no change. The user further is advised at 2406 of the date and time for which no change has occurred in the calculated early warning score; as shown in FIG. 24, the user is advised that no change has occurred since 5:15 on Jun. 27, 2014.

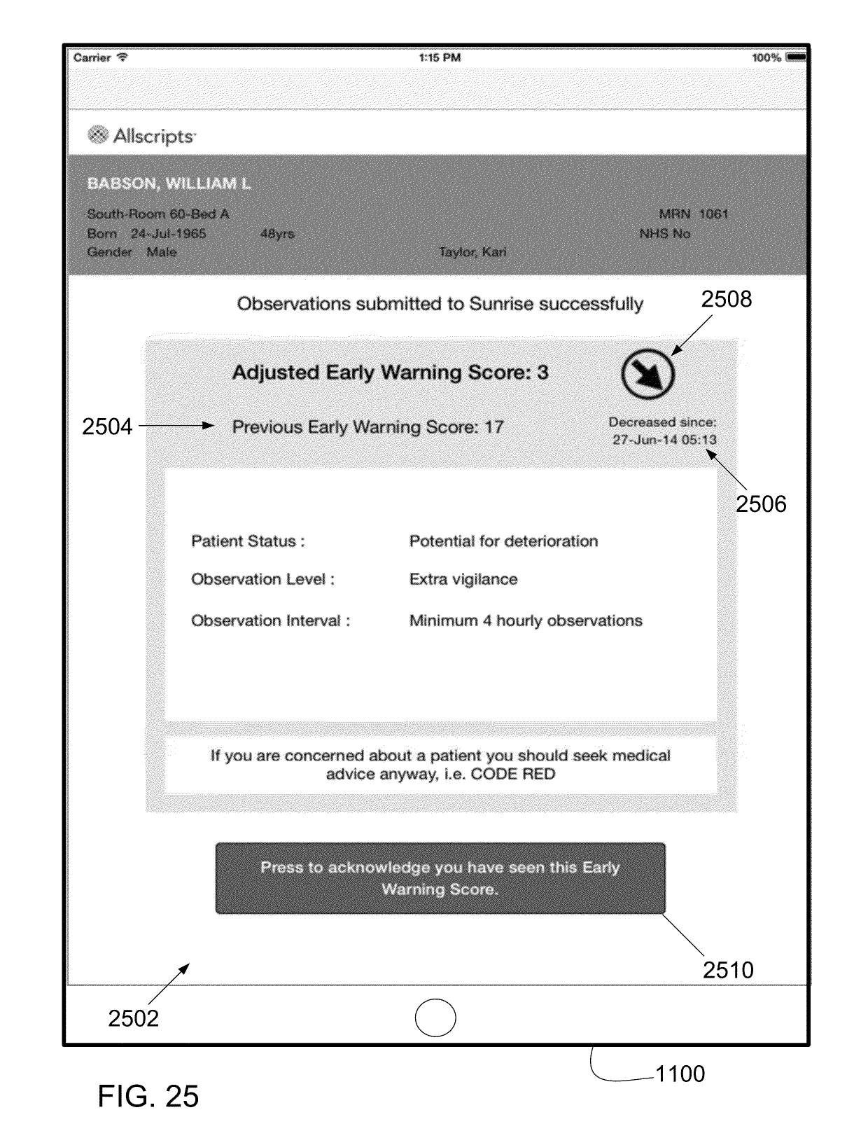

FIG. 25 illustrates the display to a user on iOS mobile device 1100 of a user interface 2502 for an early warning score calculated following submission, wherein the score is calculated to be a “3”. This indicates that the patient's status is “Potential for deterioration”; that the observation level is “Extra vigilance”; and that observations should be made at intervals of a minimum of four hours. The user interface 2502 includes a “yellow” theme indicating the patient status and cautious early warning score that has been calculated. A similar user interface is displayed for a calculated score of 4.

If an early warning score has been previously calculated, as recorded in the EPR for the patient, then the user interface 2502 further displays the early warning score at 2504, which here was previously calculated to be “17”; further displays that the early warning score is trending lower at 2506; and further displays a visual indication of the decreasing trend in the score at 2508; as shown here the “arrow” in the circle is pointing downward indicating that the score is trending lower. The user further is advised at 2506 of the date and time of the previous calculated score, from which the trend in the score is determined; as shown in FIG. 25, the user is advised that the score has decreased since 5:13 on Jun. 27, 2014.

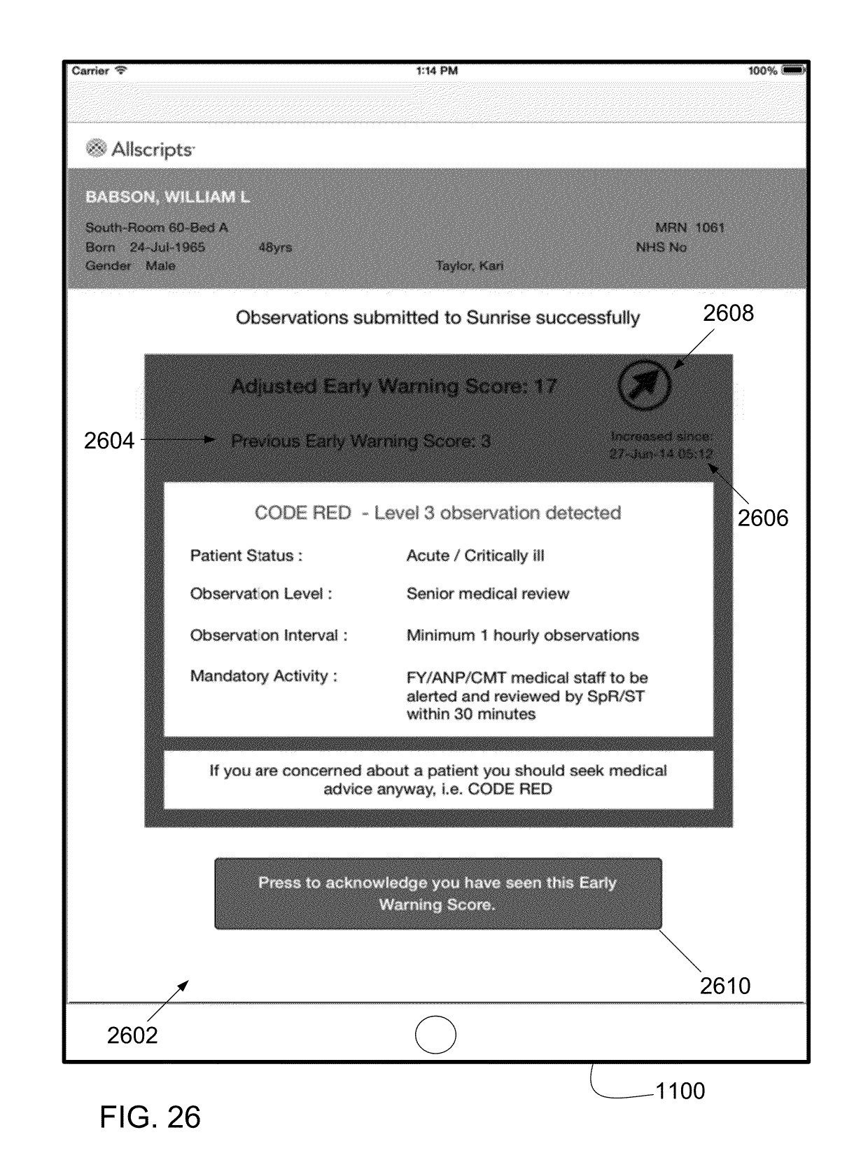

FIG. 26 illustrates the display to a user on iOS mobile device 1100 of a user interface 2602 for an early warning score calculated following submission, wherein the score is calculated to be a “17”. This indicates that the patient's status is “Acute/Critically ill”; that the observation level requires “Senior medical review”; and that observations should be made at intervals of a minimum of one hour. The user interface 2602 further advises the user of mandatory activity, including “FY/ANP/CMT medical staff to be alerted and reviewed by SpR/ST within 30 minutes”. The user interface 2602 includes a “red” theme indicating the critical and urgent status of the patient based on the early warning score that has been calculated. A similar “red” user interface with any warranted mandatory activity are displayed for calculated scores of 7 or more. For further emphasis, the label “CODE RED—Level 3 observation detected” also is displayed in the user interface 2602.

If an early warning score has been previously calculated, as recorded in the EPR for the patient, then the user interface 2602 further displays the early warning score at 2604, which here was previously calculated to be “3”; further displays that the early warning score is trending higher at 2606; and further displays a visual indication of the increasing trend in the score at 2608; as shown here the “arrow” in the circle is pointing upward indicating that the score is trending higher. The user further is advised at 2606 of the date and time of the previous calculated score, from which the trend in the score is determined; as shown in FIG. 26, the user is advised that the score has increased since 5:12 on Jun. 27, 2014.

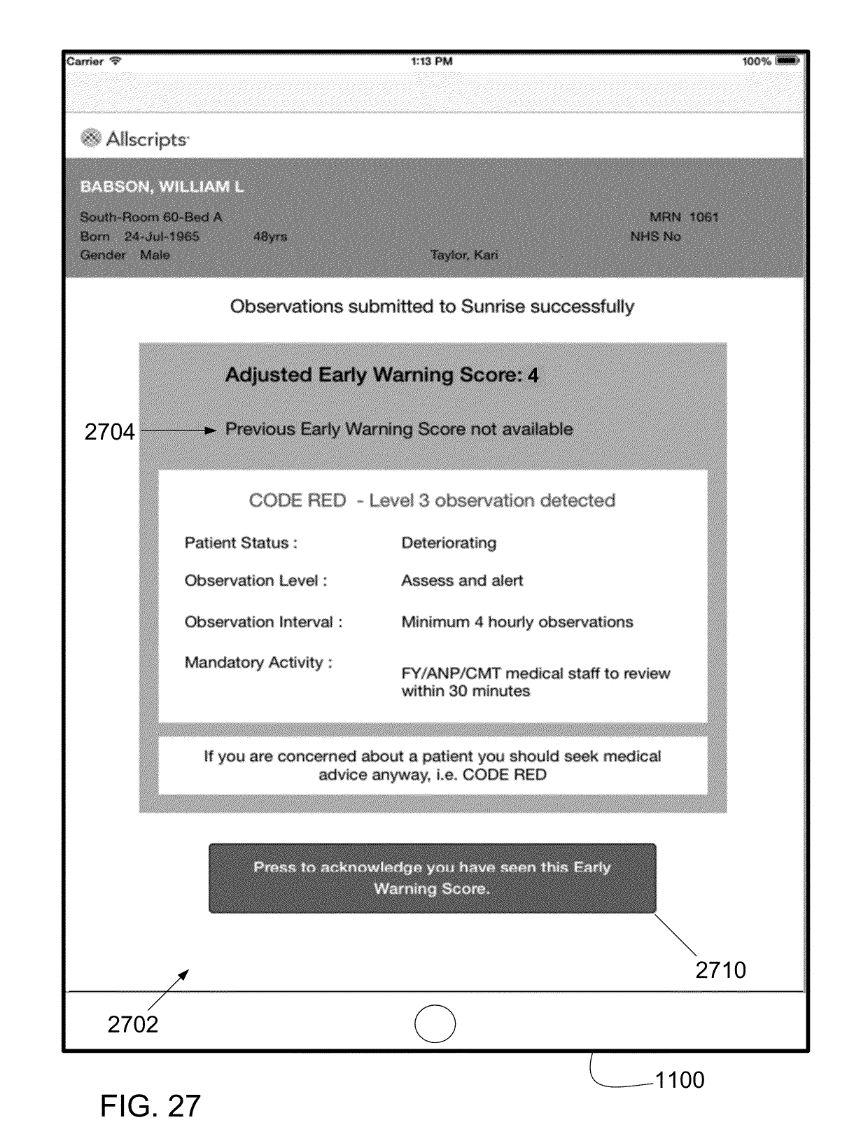

FIG. 27 illustrates the display to a user on iOS mobile device 1100 of a user interface 2702 for an early warning score calculated following submission, wherein the score is calculated to be a “4”, which indicates that the patient's status is “Deteriorating”; that the observations level requires “Access and alert”; and that observations should be made at intervals of a minimum of four hours. The user interface 2702 further advises the user of mandatory activity, including “FY/ANP/CMT medical staff to review within 30 minutes”. The user interface 2702 includes an “orange” theme indicating the close-to-critical and potentially urgent status of the patient based on the early warning score that has been calculated. A similar “orange” user interface with any warranted mandatory activity is displayed for calculated scores of 5 and 6. For further emphasis, the label “CODE RED—Level 3 observation detected” is displayed in the user interface 2702.

It will further be appreciated that, in this example, an early warning score is not available, e.g., has not been previously recorded in the EPR for the patient, and thus the user interface 2702 does not display any information related thereto or based thereon.

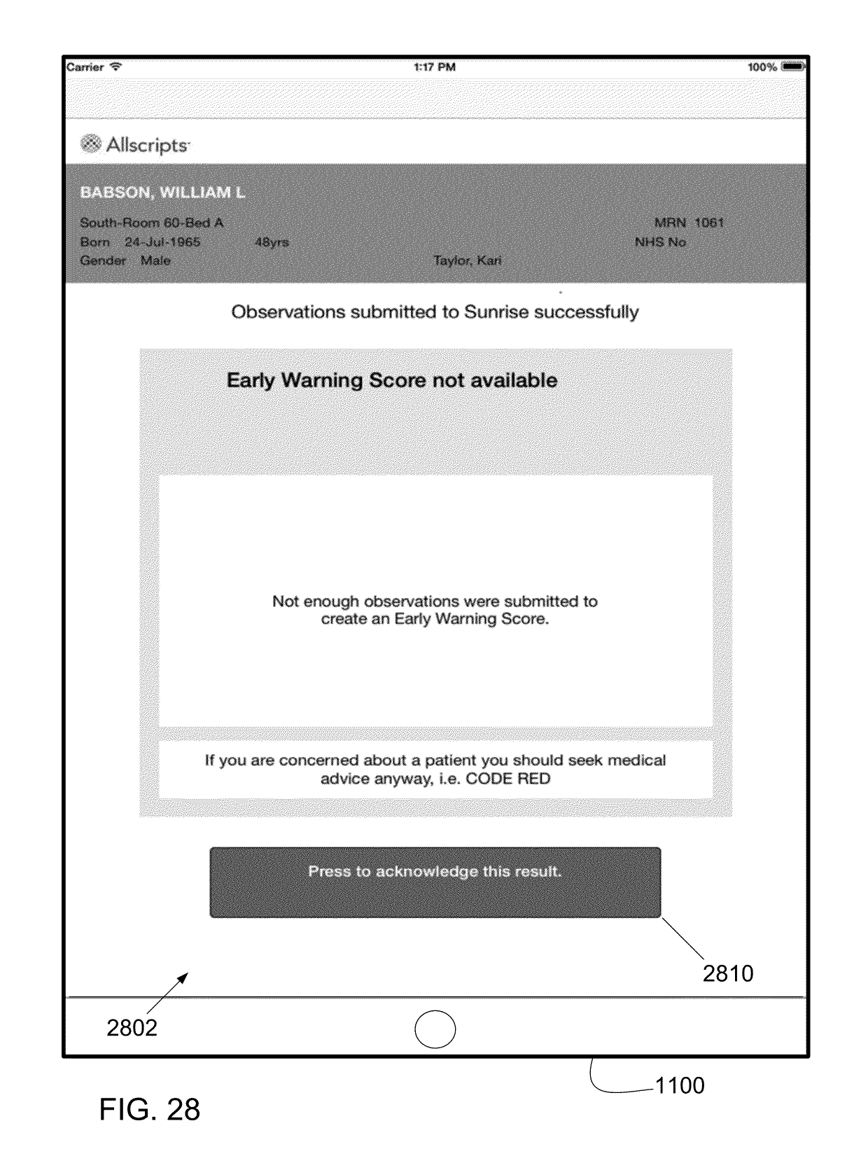

FIG. 28 illustrates the display to a user on iOS mobile device 1100 of a user interface 2802 wherein not enough observations have been made contemporaneously with the submission so as to be able to calculate a current early warning score.

With reference to each of the user interfaces 2402,2502,2602, 2702,2802,2902, it will be appreciated that the user must acknowledge viewing of the respective user interface before further interacting with the application on the iOS mobile device 1100 by selecting respective button 2410,2510,2610,2710,2810,2910. This confirms that the user is alerted to, inter alia, any mandatory action that may be required, as well as to the observation level and any changes thereto.

FIG. 29 illustrates a portion of a screen shot of a user interface 2902 of an EHR Application, wherein an “Orders” tabbed page 2904 is displayed to a user that includes an “Order Summary” for a particular patient. As shown, an exception order is revealed for Nursing that relates to the calculation of an early warning score for the particular patient. In accordance with the one or more preferred embodiments of the invention, the calculation of the early warning score of the particular patient per the exception order takes into account the fact that the particular patient is an athlete with a known low resting pulse rate.

In accordance with one or more preferred implementations, asynchronous web service calls are utilized to submit observations to an EHR. Aspects and features of one or more preferred methodologies of managing asynchronous web service calls for submitting observations to an EHR will now be described.

In accordance with one or more preferred implementations for use in a medical software context, a patient observations submission state engine uses native asynchronous web service handling (e.g. that in iOS) and manages multiple submission transaction requests for a single patient observation session. Preferably, the transactions and their results are managed by the engine, and appropriate result events (success, partial failure and full failure) are pushed back to a user interface.

Preferably, partial transaction failures are handled by the user interface, providing the end user the ability to retry just the failed transactions, or ignore and continue to the final result page. This ensures safety of the transactions back into a clinical manager flow sheet (such as a flow sheet for Sunrise Clinical Manager, available from Allscripts Healthcare Solutions, Inc.), and reduces the workload of clients after an incident such as temporary connectivity issues.

Preferably, the state engine uses the publish-subscribe (also known as observer) pattern to provide segregation of responsibility, which provides clean, testable and maintainable code, thereby improving reliability and reducing maintenance costs.

One or more preferred implementations provide a code clean approach to controlling the complexity of a single user submission, triggering multiple web service transactions, and handing potential failures appropriately (for example, 1st and 3rd calls are successful, but the 2nd fails).

In one or more preferred implementations, a state engine has a configurable pause feature, which allows the engine to pause between transactions, notifying the user, and waiting on a user trigger (e.g. button press) to continue. The pause feature preferably provides a test user the ability to control the success or failure of a given transaction, and thus test all scenarios reliably, thereby improving the quality of an end product.

One or more preferred implementations provide a means for a test user to reliably test each possible scenario, by pausing the process (by configuration) between web service submissions in order to control the success or failure of the web service call, and thus test the expected behavior of the app.

One or more preferred implementations utilize a publish-subscribe pattern and provide clean separation of 3 tiers within an application—a user interface, a submission management engine, and web service call handling. Preferably, each tier has limited or no real understanding of the others, and thus provides segregated testable code, which in turn improves the quality of the product, and reduces maintenance costs.

In accordance with one or more preferred implementations, a publish and subscribe pattern (also known as the observer pattern) is central to the segregation of responsibility within. There are other ways to manage the asynchronous nature of web service calls within an OS such as iOS, however they blur the lines of segregation, each tier needing to know too much about the others. Preferably, the pattern also makes the use of pausing between transactions much simpler, allowing the user interface to respond to a variety of outcomes (in this case pause, fail, partial fail and success) with no knowledge of the logic to determine what the outcome will be.

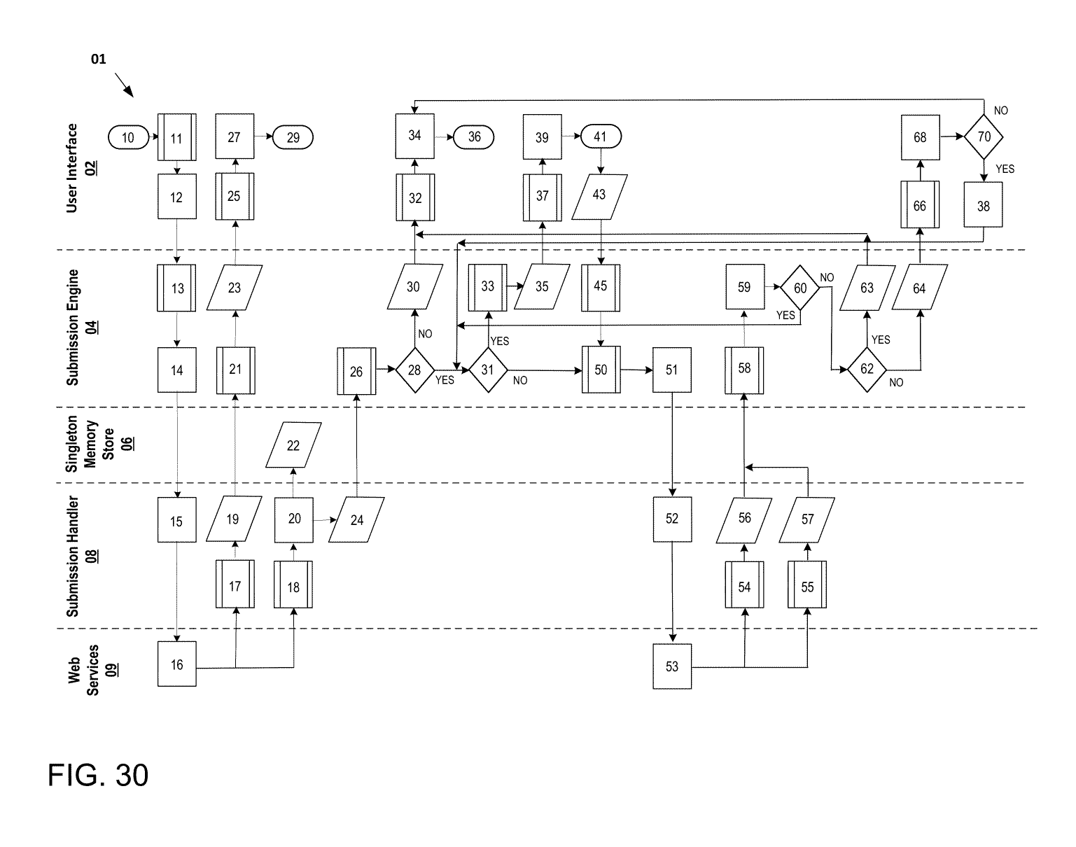

A preferred method of managing asynchronous web service calls for submitting observations to an EHR is now described with reference to FIG. 30.

With reference now to FIG. 30, a flowchart is shown that illustrating a sequence 01 of steps performed in accordance with a preferred embodiment of the invention. The flowchart pertains to the submission into a Sunrise Clinical Manager (SCM) workflow of patient observations made and, in the example, to the submission of observations including multiple blood pressures for a patient.

The sequence 01 begins at step 10, when a ‘submit’ button of a display of a user interface 02 displayed on an iOS device, such as an iPad, is selected by a user. This effects, at step 11, registration as a subscriber (in accordance with a publish-subscribe pattern) to messages concerning the submission from a submission engine. At step 12, the UI triggers a submission.

The submission engine 04, based on the triggering of the submission, in turn at step 13 registers as a subscriber to receive messages regarding a call concerning the submission, and at step 14 submits primary observations (e.g. including a blood pressure measurement).

A submission handler 08 responds at 15 by generating a call to web services 09.

The web services 09 handles the call at 16.

Depending on the results of the handling of the call by the web services 09, the submission handler 08 handles a “failure”, “timeout” or “success with invalid response” at 17, publishing a “call failed” message at 19. The submission engine 04 handles at 21 the “call failed” message published by the submission handler 08, and in turn the submission engine 04 publishes a failure message at 23. The user interface 02 handles at 25 the failure message published by the submission engine 04, and notifies the user of the failure at 27, after which performance of the sequence 01 ends at 29 for the particular patient observation session.

Alternatively, the submission handler 08 handles a “success with valid response” at 18, adding result details to a singleton memory store at 20; storing the submission result at 22; and publishing a call success message at 24. The submission engine 04 handles at 26 the call success message published by the submission handler 08 and then determines at 28 whether an observation comprising a next blood pressure is to be communicated to the web services 09.

If there is not a next blood pressure to be communicated, then the submission engine 04 publishes a success message at 30. The user interface 02 handles at 32 the publication of the success message by the submission engine 04, and displays an indication of success at 34, after which performance of the sequence 01 ends at 36 for the particular patient observation session.

If there is a next blood pressure, then the submission engine 04 determines at 31 whether a “test pause” is active. If active, the submission engine registers as a pause subscriber at 33, and publishes a “test pause” message at 35. The user interface 02 handles at 37 the publication of the “test pause” message by the submission engine 04, and causes a “pause” modal window to be shown at 39. The modal window persists until a user clicks “OK” to continue at 41, upon which a “pause continue” message is published at 43 by the user interface 02.

The submission engine 04 handles at 45 the publication by the user interface 02 of the “pause continue” message, whereupon the submission engine again registers as a call subscriber at 50 and submits an additional observation (e.g. including a next blood pressure measurement) at 51. The submission handler responds at 52 by generating a call to the web services 09, and the web services 09 handles the call at 53.

Depending on the results of the handling of the call by the web services 09, the submission handler 08 handles a “failure”, “timeout” or “success with invalid response” at 54, publishing a “call failed” message at 56. Alternatively, the submission handler 08 handles a “success with valid response” at 55, publishing a “call success” message at 57.

The submission engine 04 handles at 58 the result (either the publication of the “call failed” message or the publication of the “call success” message for the additional observation submitted), and the submission result is logged at 59. The submission engine 04 then determines at 60 whether there is an additional observation comprising a next blood pressure for submission. If there is a next blood pressure, then the flow returns to the determination at 31 as to whether a “test pause” is active.

If there is not a next blood pressure, then a determination is made at 62 whether all calls for additional observation submissions to the web services 09 have been successful. If all are successful, then the submission engine 04 publishes at 63 a “success” message. The user interface 02 handles at 32 the publication of the “success” message by the submission engine 04, and displays an indication of success at 34, after which performance of the sequence 01 ends at 36 for the particular patient observation session.

On the other hand, if all are not successful, then the submission engine 04 publishes notification of a partial success at 64. The user interface 02 handles at 66 the publication of the “partial success” message by the submission engine 04, and informs the user at 68 of the failed additional submission(s). A determination at 70 is then made, based on received user input, regarding whether the failed submission(s) are to be attempted again. If no reattempt is to be made, then the user interface 02 displays an indication of the (partial) success at 34, after which performance of the sequence 01 ends at 36 for the particular patient observation session. If the failed submission(s) are to be attempted again, then the flow returns to the determination at 31 as to whether a “test pause” is active, with the observation(s) comprising the blood pressure(s) of the failed submission(s) representing the next respective blood pressures to be submitted.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

1. A computer network for providing early warning scores for a plurality of patients, which scores are used by medical personnel to identify degrees of illness or injury of patients, the computer network comprising,
one or more networked desktop computers and one or more mobile computing devices; and
a server system comprising one or more computers and arranged in electronic communication with the one or more networked desktop computers and one or more mobile computing devices;
wherein the server system includes a non-transient computer readable medium in which an electronic health records (EHR) application is stored in the form of computer-executable code, by which EHR application each of the computers and mobile computing devices can access, over the computer network, patient data in electronic patient records maintained for patients;
wherein the server system includes a non-transient computer readable medium in which one or more logic modules are stored, each logic module being accessible by the EHR application and each logic module providing logic by which a task is performed by or for the EHR application, the logic of each logic module being alterable without alteration to the computer-executable code of the EHR application; and
wherein one of the one or more logic modules provides logic by which an early warning score is calculated, the logic including calculating an early warning score based on, at least in part, data from an electronic patient record that is accessed by the EHR application for calculating such early warning score;
wherein the EHR application is adapted to communicate a calculated early warning score to at least one of the one or more networked devices, which in turn is adapted to display the calculated early warning on a screen for view by a user.

2. The method of claim 1, wherein the data from the electronic patient record on which data the early warning score is calculated includes a patient respiration rate.

3. The method of claim 1, wherein the data from the electronic patient record on which data the early warning score is calculated includes an oxygen saturation.

4. The method of claim 1, wherein the data from the electronic patient record on which data the early warning score is calculated includes an oxygen device flow rate.

5. The method of claim 1, wherein the data from the electronic patient record on which data the early warning score is calculated includes a temperature of the patient.

6. The method of claim 1, wherein the data from the electronic patient record on which data the early warning score is calculated includes a blood pressure of the patient.

7. The method of claim 1, wherein the data from the electronic patient record on which data the early warning score is calculated includes a heart rate of the patient.

8. The method of claim 1, wherein the data from the electronic patient record on which data the early warning score is calculated includes a level of consciousness of the patient.

9. The method of claim 1, wherein the data from the electronic patient record on which data the early warning score is calculated includes a level of pain experienced by the patient.

10. The computer network of claim 1, wherein the logic module is configured to calculate the early warning scores.

11. The computer network of claim 1, wherein the EHR application is configured to calculate the early warning scores using the logic provided by the logic module.

12. The computer network of claim 1, wherein the computer network is implemented by a plurality of healthcare facilities, and wherein a single logic module provides different logic for calculating early warning scores for patients of at least two of the healthcare facilities based on identification of the healthcare facility of a patient.

13. The computer network of claim 1, wherein the computer network is implemented by a plurality of medical services departments of a healthcare facility, and wherein a single logic module provides different logic for calculating early warning scores for patients of at least two of the medical services departments based on identification of the medical services department of a patient.

14. The computer network of claim 1, wherein a single logic module provides different logic for calculating early warning scores for patients based on an identification of a patient group of a patient from a plurality of different patient groups.

15. The computer network of claim 1, wherein a single logic module provides different logic for calculating early warning scores for patients based on an identification of an EWS standard from a plurality of different EWS standards.

16. The computer network of claim 1, wherein the computer network is implemented by a plurality of healthcare facilities, and wherein the computer network further comprises a plurality of logic modules each corresponding to a respective one of the plurality of healthcare facilities, at least two of the logic modules providing different logic for calculating early warning scores for patients based on the healthcare facility of the patient.

17. The computer network of claim 1, wherein the computer network is implemented by a plurality of medical service departments of a healthcare facility, and wherein the computer network further comprises a plurality of logic modules each corresponding to a respective one of the medical services departments of the healthcare facility, at least two of the logic modules providing different logic for calculating early warning scores for patients based on the medical service department of the patient.

18. The computer network of claim 1, wherein the computer network further comprises a plurality of logic modules each corresponding to a respective patient group, at least two of the logic modules providing different logic for calculating early warning scores for patients.

19-28. (canceled)

29. A method of calculating in a computer network early warning scores for a plurality of patients, which scores are used by medical personnel to identify degrees of illness or injury of patients, the method comprising:
(a) receiving from networked devices for a plurality of patients, by an electronic health records (EHR) application, observation data for one or more observations made regarding the patients, and for each of the patients, recording in an electronic patient record of such patient the received observation data for such patient;
(b) accessing from a logic module, by the EHR application, logic for calculating an early warning score and, in accordance with such logic, accessing recorded observation data from an electronic patient record for calculating the early warning score based on the accessed observation data recorded in the electronic patient record; and
(c) communicating, by the EHR application, to one of the networked devices, for display to medical personnel, an early warning score calculated based on the accessed logic and accessed observation data recorded in the electronic patient record.

30-47. (canceled)

48. A method for quickly and accurately identifying a degree of illness or injury of a patient, comprising:
(a) receiving, by a networked device, user input regarding one or more observations made regarding the patient;
(b) communicating, by the networked device, the received user input to an electronic health records (EHR) application for recording thereof in an electronic patient record of the patient;
(c) in response to performance of said step (b) by the networked device,
(i) receiving, by the networked device, from the EHR application, a current early warning score for the patient based at least in part on the communicated user input when sufficient user input regarding one or more observations has been communicated for calculating a current early warning score for the patient, and
(i) receiving, by the networked device, from the EHR application, a message that the user input communicated was insufficient for calculating a current early warning score for the patient when insufficient user input regarding one or more observations has been communicated for calculating a current early warning score for the patient; and
(d) displaying, by the networked device for view by a user,
(i) the current early warning score when received from the EHR application, and
(ii) the message that the user input communicated was insufficient for calculating a current early warning score for the patient when such message is received from the EHR application.

49-91. (canceled)
